(19) United States
(12) Patent Application Publication
(10) Pub. No.: US 2026/0182921 A1
(43) Pub. Date: Jul. 2, 2026

(54) DOCKING STRUCTURES, DOCKING METHODS, DOCKING DETACHMENT STRUCTURES, AND FLOATING STRUCTURES

(71) Applicant: SHANGHAI UNITED IMAGING HEALTHCARE CO., LTD., Shanghai (CN)

(72) Inventors: Yang ZHANG, Shanghai (CN); Wei ZHANG, Shanghai (CN)

(73) Assignee: SHANGHAI UNITED IMAGING HEALTHCARE CO., LTD., Shanghai (CN)

(21) Appl. No.: 19/548,943

(22) Filed: Feb. 24, 2026

Related U.S. Application Data
(63) Continuation of application No. PCT/CN2024/114638, filed on Aug. 26, 2024.

(30) Foreign Application Priority Data
Aug. 31, 2023 (CN) 202311120530.5
Aug. 31, 2023 (CN) 202322368493.1
Aug. 31, 2023 (CN) 202322371631.1

(51) Int. Cl. A61B 5/00; A61B 5/055

(52) U.S. Cl. CPC A61B 5/704; A61B 5/055; A61B 5/742

(57) ABSTRACT

A docking structure, docking method, docking detachment structure, and floating structure are disclosed. The docking structure may be installed on a medical bed and configured to dock with a connection structure of an imaging device to achieve docking between the medical bed and the imaging device. The docking method may include: driving the medical bed to move towards the imaging device to a first critical position; driving the medical bed to continue moving to a second critical position; driving the medical bed to continue moving to a third critical position; and initiating a electric driving assembly to drive a mounting seat to move relative to the medical bed, so that the medical bed docks with the imaging device. The docking detachment structure is fit with the docking structure and may be provided on the connection structure. The floating structure may be used for a bed-side connector of the docking structure and/or for an imaging-side connector of the connection structure of the imaging device.

### CROSS-REFERENCE TO RELATED APPLICATIONS

This application is a continuation of International Application No. PCT/CN 2024/114638, filed on Aug. 26, 2024, which claims priority to Chinese Patent Application No. 202311120530.5, filed on Aug. 31, 2023, Chinese Patent Application No. 202322368493.1, filed on Aug. 31, 2023, and Chinese Patent Application No. 202322371631.1, filed on Aug. 31, 2023, the entire contents of each of which are incorporated herein by reference.

### TECHNICAL FIELD

The present disclosure relates to the field of medical devices, and particularly to docking structures, docking methods, docking detachment structures, and floating structures.

### BACKGROUND

Medical imaging equipment plays an increasingly important role in the diagnosis and treatment of diseases. A medical imaging equipment includes an imaging device and a medical bed. In clinical operations, to facilitate scanning and imaging of a patient, a movable medical bed is used to transport the patient to the imaging device, and then a bed board of the medical bed is moved to transfer the patient into an imaging channel for imaging. Most movable medical beds have a docking structure that enables electrical and/or data connection between the medical bed and the imaging device to ensure precise positioning of the medical bed, so that the patient on the medical bed can be accurately positioned within the imaging channel of the imaging device.

Currently, the docking between the docking structure of the medical bed and a connection structure of the imaging device is generally achieved through hydraulic drive. However, hydraulic drive systems are expensive and bulky, and their operational comfort is relatively poor.

Therefore, it is desirable to provide a docking structure, docking method, docking detachment structure, and floating structure to enhance the reliability of the docking between the medical bed and the imaging device, reduce the difficulty of docking, and improve operational comfort.

### SUMMARY

One or more embodiments of the present disclosure provide a docking structure installed on a medical bed. The docking structure may be configured to dock with a connection structure of an imaging device to achieve docking between the medical bed and the imaging device. The docking structure comprises an electric driving assembly, the electric driving assembly being provided on the medical bed, and the electric driving assembly being at least configured to drive the medical bed to move towards the imaging device, so as to achieving docking between the docking structure and the connection structure, and achieving docking between the medical bed and the imaging device.

In some embodiments, the docking structure may include a mounting seat. A tensioning part may be provided on the mounting seat. The tensioning part may have a locked position and an unlocked position, and the tensioning part may be configured to fit with a limiting part of the connection structure to lock or unlock a movement of the mounting seat. An elastic part may be provided between the tensioning part and the mounting seat, the elastic part being configured to restore the tensioning part from the unlocked position to the locked position. The docking structure may further include a bed-side connector, the bed-side connector being provided at one end of the medical bed that docks with the imaging device, and the electric driving assembly being drivingly connected to the mounting seat.

In some embodiments, the electric driving assembly may be at least configured to: when the tensioning part is in the locked position and cooperates with the limiting part of the connection structure, the electric driving assembly drives the medical bed to move towards the mounting seat, so as to cause the medical bed to move relative to the mounting seat, thereby achieving docking between the bed-side connector and the connection structure, and achieving docking between the medical bed and the imaging device.

In some embodiments, the docking structure may include a tensioning assembly, which may include a rotating shaft provided on the mounting seat. A pull rod may be rotatably connected to the rotating shaft, and one end of the pull rod may be provided with the tensioning part.

In some embodiments, the docking structure may further include a supporting part. The supporting part may be connected to the medical bed, the bed-side connector may be provided on the supporting part, a stopping part may be provided on the supporting part, and a projection of the stopping part may at least partially overlap with an end of the pull rod away from the tensioning part in a first direction, the first direction being a length direction of the medical bed.

In some embodiments, the tensioning assembly may include two pull rods arranged at interval in a second direction, the second direction being a width direction of the medical bed. The two pull rods may be rotatably connected to the rotating shaft, and the two pull rods may be provided with the tensioning part, respectively. Each end of the two pull rods away from the corresponding tensioning part may be connected by a connecting rod, and the projection of the stopping part may at least partially overlap with the connecting rod in the first direction.

In some embodiments, the electric driving assembly may include a driving source. In a docking state, the driving source is arranged at a position where a magnetic field strength of a magnet of the imaging device is 5 mT-200 mT.

In some embodiments, the electric driving assembly may further include a moving structure, the driving source may be arranged on the moving structure, and the moving structure may be configured to adjust a position of the driving source, thereby the driving source is arranged at positions corresponding to different magnetic field strengths of the magnet of the imaging device in the docking state.

In some embodiments, in the docking state, the distance between the driving source and the magnet facing one side of the medical bed may be positively correlated with a maximum magnetic field strength of the magnet.

In some embodiments, the docking structure may be provided with a sensor, the sensor being configured to measure an orientation of the docking structure relative to the connection structure of the imaging device, the orientation including a direction and/or a distance of the docking structure relative to the connection structure of the imaging device.

In some embodiments, the sensor may be connected to a display device via a signal, the display device being configured to display a measurement result of the sensor.

In some embodiments, the docking structure may further include a guiding part connected to the bed-side connector, the guiding part may be configured to fit with a guidance part of the connection structure to guide the docking of the bed-side connector with an imaging-side connector of the connection structure.

In some embodiments, the docking structure may be detachably connected to the medical bed.

In some embodiments, the bed-side connector may further include a height adjustment mechanism, the height adjustment mechanism may be configured to adjust a height of the bed-side connector relative to the connection structure of the imaging device.

One or more embodiments of the present disclosure provides a docking method for an imaging device and a medical bed including a docking structure of described in any embodiment of the present disclosure. The docking method may inlcude: driving the medical bed to move towards the imaging device to a first critical position. When the medical bed is at the first critical position, a limiting part of the connection structure just contacts a tensioning part of the docking structure, and the tensioning part is in a locked position; driving the medical bed to continue moving to a second critical position. When the tensioning part gradually moves from the locked position to an unlocked position, a elastic part of the docking structure gradually stretches, and when the medical bed is at the second critical position, the tensioning part is in the unlocked position; driving the medical bed to continue moving to a third critical position The tensioning part moves across the limiting part and is restored from the unlocked position to the locked position under an action of the elastic part, and when the medical bed is at the third critical position, the tensioning part hooks with the limiting part; and initiating the electric driving assembly to drive a mounting seat of the docking structure to move relative to the medical bed, so that the medical bed docks with the imaging device.

In some embodiments, the docking structure and/or the medical bed may be provided with a sensor, the medical bed may be provided with a processor, and the driving the medical bed to move towards the imaging device to the first critical position may include: automatically adjusting a movement parameter of the medical bed by the processor based on a measurement result of the sensor.

In some embodiments, the movement parameter of the medical bed may include at least one of a moving direction, a moving speed, or a height of the medical bed relative to the ground.

In some embodiments, the bed-side connector further may include a height adjustment mechanism, and the driving the medical bed to move towards the imaging device to the first critical position may include: controlling, by the processor, based on the measurement result of the sensor, the height adjustment mechanism to adjust a height of the bed-side connector.

In some embodiments, when the medical bed is at the third critical position, the tensioning part may hook with the limiting part to lock the movement of the mounting seat, and the initiating the electric driving assembly to drive the mounting seat to move relative to the medical bed, so that the medical bed docks with the imaging device may include: driving the mounting seat to move in a direction away from the imaging device by the electric driving assembly, so that a driving force acting on the mounting seat reacts against the medical bed, causing the medical bed to move towards the imaging device to complete the docking.

In some embodiments, the tensioning part may be provided with a signal generator. The signal generator may be configured to output a sensing signal when the tensioning part hooks with the limiting part, and the initiating the electric driving assembly to drive the mounting seat to move relative to the medical bed, so that the medical bed docks with the imaging device may include: in response to receiving the sensing signal, initiating the electric driving assembly.

In some embodiments, the docking structure may be detachably connected to the medical bed, and the docking method may further include: connecting the docking structure to the medical bed.

One or more embodiments of the present disclosure provide a docking detachment structure that is fit with a docking structure described in any embodiment of the present disclosure. The docking detachment structure may be provided on the connection structure, the docking detachment structure is configured such that: under an action of external force, the docking detachment structure causes the docking structure and the connection structure to separate along a first direction, and a direction of the external force is perpendicular to the first direction.

In some embodiments, the docking detachment structure may include: a docking base; a sliding part slidably connected to the docking base, the sliding part being provided with the limiting part; a first elastic member connecting between the docking base and the sliding part; and an operating part rotatably connected to the docking base about an axis in the first direction, one end of the operating part abutting the sliding part. The operating part drives the sliding part to move along a second direction under the action of the external force, so as to allow the docking structure to separate from the connection structure along the first direction.

One or more embodiments of the present disclosure provide a floating structure for a bed-side connector of a docking structure described in any embodiment of the present disclosure and/or for an imaging-side connector of a connection structure of an imaging device. The floating structure may include: a cone portion, an elastic structure, a first plate, and a second plate. The first plate may be provided with a through-hole, the cone portion may abut against the through-hole, the elastic structure may be located between the first plate and the second plate, an outer peripheral surface of the cone portion may taper along a direction of insertion into the through-hole, and an elastic coefficient of the elastic structure may be a variable value.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram of an exemplary application scenario of a medical imaging system according to some embodiments of the present disclosure;

FIG. 2 is a schematic diagram of an exemplary structure of a medical imaging system according to some embodiments of the present disclosure;

FIG. 3 is a schematic diagram of an exemplary structure of a connection structure according to some embodiments of the present disclosure;

FIG. 4 is a schematic diagram of an exemplary structure of a docking structure according to some embodiments of the present disclosure;

FIG. 5 is a schematic diagram of another perspective of the docking structure shown in FIG. 4;

FIG. 6A is a schematic diagram of a medical bed in a first critical position according to some embodiments of the present disclosure;

FIG. 6B is a schematic diagram of a medical bed in a second critical position according to some embodiments of the present disclosure;

FIG. 6C is a schematic diagram of a medical bed in a third critical position according to some embodiments of the present disclosure;

FIG. 6D is a schematic diagram of a separation of a docking structure and a connection structure according to some embodiments of the present disclosure;

FIG. 7 is a flowchart of an exemplary docking process between a medical bed and an imaging device according to some embodiments of the present disclosure;

FIG. 8 is a schematic diagram of an exemplary structure of a connection structure according to some embodiments of the present disclosure;

FIG. 9 is a schematic diagram of another perspective of the connection structure shown in FIG. 8 according to some embodiments of the present disclosure;

FIG. 10 is a schematic diagram of an exemplary structure of a floating structure according to some embodiments of the present disclosure;

FIG. 11 is a schematic diagram of another exemplary structure of the floating structure according to some embodiments of the present disclosure;

FIG. 12 is a schematic diagram of another exemplary structure of the floating structure according to some embodiments of the present disclosure;

FIG. 13 is a schematic diagram of another exemplary structure of the floating structure according to some embodiments of the present disclosure;

FIG. 14 is a schematic diagram of another exemplary structure of the floating structure according to some embodiments of the present disclosure;

FIG. 15 is a schematic diagram of an exemplary structure of a floating connector according to some embodiments of the present disclosure; and

FIG. 16 is a schematic diagram of an exemplary structure of another floating connector that mates with the floating connector shown in FIG. 15.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Flowcharts are used in the present disclosure to illustrate the operations performed by the system according to some embodiments of the present disclosure. It should be understood that the operations described herein are not necessarily executed in a specific order. Instead, the operations may be executed in reverse order or simultaneously. Additionally, one or more other operations may be added to these processes, or one or more operations may be removed from these processes.

Currently, the docking between a docking structure of a medical bed and a connection structure of an imaging device is generally achieved through hydraulic drive. However, hydraulic drive systems are expensive and bulky, and their operational comfort is relatively poor. When the docking is driven by a motor, the motor is typically installed on the imaging device. A magnetic field of the motor may interfere with a magnet of the imaging device, necessitating an additional shimming device or requiring specific orientations for the motor (e.g., the motor needs to be parallel to a direction of the magnetic field), resulting in a complex structure.

Some embodiments of the present disclosure address these issues by placing an electric driving assembly on the medical bed. This configuration ensures that, when the medical bed docks with the imaging device, a driving source (e.g., the motor) is farther away from the magnetic field of the magnet of the imaging device, thereby reducing interference between the driving source (e.g., the motor) and the magnetic field. Consequently, the driving source (e.g., the motor) does not need to be aligned parallel to the direction of the magnetic field, allowing for a flexible structural design. Moreover, the magnet of the imaging device does not require additional shimming devices, reducing structural complexity and material costs. Additionally, using the electric driving assembly enhances the reliability of the docking between the medical bed and the imaging device, thereby improving operational comfort. Specifically, the medical bed may be driven towards the imaging device until a limiting part and a tensioning part just come into contact, with the tensioning part in a locked position. The medical bed may be continue driven forward, causing the tensioning part to gradually move past the limiting part, and transit from the locked position to an unlocked position. An elastic part stretches gradually. When the tensioning part is about to pass but has not yet passed the limiting part, the elastic part reaches its maximum extension, thus the tensioning part in the unlocked position. The medical bed may be further driven to cause the tensioning part to move past the limiting part, and the tensioning part then returns to the locked position under an action of the elastic part, such that the tensioning part hooks with the limiting part. At this point, a mounting seat may be connected and relatively fixed to the connection structure. The electric driving assembly may be initiated to drive the medical bed towards the mounting seat, thus docking the docking structure with the connection structure and completing the docking of the medical bed with the imaging device.

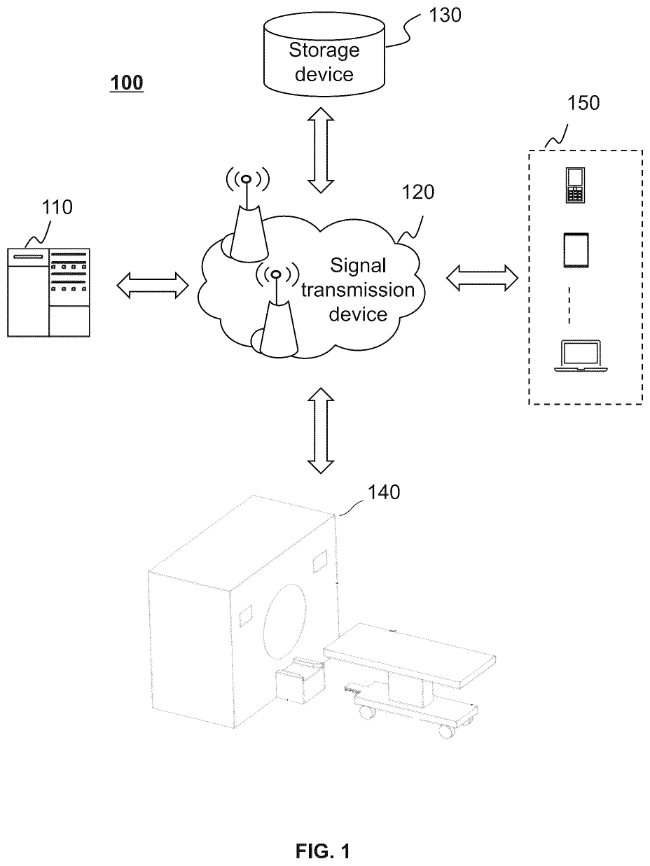

FIG. 1 is a schematic diagram of an exemplary application scenario of a medical imaging system according to some embodiments of the present disclosure.

In some embodiments, an application scenario 100 of the medical imaging system may include a processor 110, a signal transmission device 120, a storage device 130, a medical imaging system 140, and one or more terminal devices 150. In some embodiments, the processor 110 may be connected to the storage device 130, the medical imaging system 140, and/or the one or more terminal devices 150 via the signal transmission device 120 to access and/or receive data and information. For example, the processor 110 may receive relevant information from the medical imaging system 140 (e.g., a measurement result of a sensor) via the signal transmission device 120. The application scenario 100 may control the operation of the medical imaging system by implementing the methods and/or processes disclosed in the present disclosure.

The processor 110 may be configured to process data and/or information from at least one component of the application scenario 100 or from an external data source (e.g., a cloud data center). The processor 110 may be connected to the storage device 130, the medical imaging system 140, and/or the one or more terminal devices 150 via the signal transmission device 120 to access and/or receive data and information. For example, the processor 110 may receive relevant information from the medical imaging system 140 (e.g., a measurement result of a sensor) via the signal transmission device 120. In some embodiments, the processor 110 may send parameters (e.g., motion parameters) related to the medical imaging system 140 to the one or more terminal devices 150 via the signal transmission device 120.

In some embodiments, the processor 110 is included in the medical imaging system 140. In some embodiments, the processor 110 may automatically adjust the motion parameters of the medical bed based on the measurement result of the sensor. In some embodiments, the processor 110 may be configured to control a height adjustment mechanism to adjust a height of a bed-side connector based on the measurement result of the sensor.

In some embodiments, the processor 110 may include one or more processing engines (e.g., single-chip or multi-chip processing engines). For example, the processor 110 may include a central processing unit (CPU). The processor 110 may process data, information, and/or processing results obtained from other devices or system components and execute program instructions based on the data, information, and/or processing results to perform one or more functions described in the present disclosure.

The signal transmission device 120 may connect the components (e.g., the storage device 130, medical imaging system 140, one or more terminal devices 150, etc.) of the application scenario 100 and/or connect the application scenario 100 with external resources. The signal transmission device 120 enables communication between components and with external parts of the application scenario 100, facilitating the exchange of data and/or information. In some embodiments, the storage device 130 may be connected to the signal transmission device 120 to communicate with one or more components (e.g., the processor 110, medical imaging system 140, one or more terminal devices 150) of the application scenario 100. In some embodiments, the signal transmission device 120 may also include a network. In some embodiments, the network may include a local area network (LAN), a wide area network (WAN), a wired network, a wireless network, etc. FIG. 1 exemplarily shows the signal transmission device 120 including a network, which is only for illustrative purposes and does not limit the embodiments of the present disclosure. It is understood that the signal transmission device 120 may transmit signals through other media. For example, the signal transmission device 120 may include a data transmission cable.

The storage device 130 may be configured to store data and/or instructions. In some embodiments, the storage device 130 may store data and/or instructions used by the processor 110 to execute or utilize exemplary methods described in the present disclosure. For example, the storage device 130 may store information output by the medical imaging system 140 (e.g., the measurement result of the sensor).

In some embodiments, the storage device 130 may be part of the processor 110. In some embodiments, the storage device 130 may include mass storage, removable storage, volatile read/write memory, read-only memory (ROM), etc. In some embodiments, the storage device 130 may be implemented on a cloud platform. In some embodiments, the storage device 130 may be connected to the signal transmission device 120 to communicate with one or more components (e.g., the processor 110, medical imaging system 140, one or more terminal devices 150) of the application scenario 100.

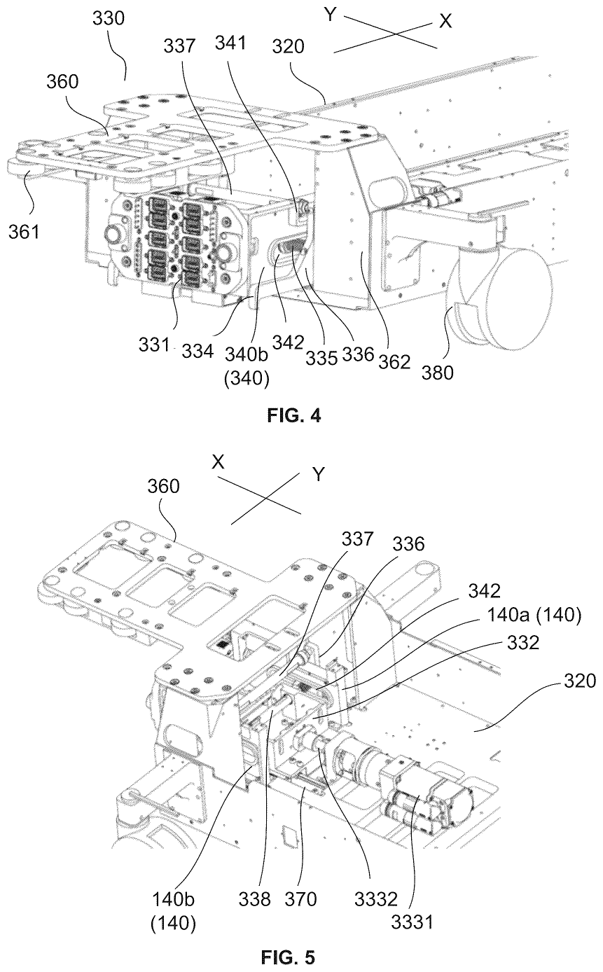

In some embodiments, the medical imaging system 140 may be used for imaging patients. In some embodiments, the medical imaging system 140 may include an imaging device (e.g., an imaging device 200) and a medical bed (e.g., a medical bed 300), with the medical bed used to carry a patient and move the patient into an imaging channel of the imaging device. The imaging device is used to image the patient in the imaging channel. The docking or separation between the medical bed and the imaging device may be achieved through the cooperation of a docking structure (e.g., a docking structure 330) installed on the medical bed and a connection structure (e.g., a connection structure 220) disposed on the imaging device. When the medical bed docks with the imaging device, the medical bed and the imaging device are connected and fixed, and there is an electrical and/or data connection between the medical bed and the imaging device. When the medical bed is detached from the imaging device, the medical bed and the imaging device are disconnected and function as separate devices. More descriptions of the docking and detachment between the medical bed and the imaging device, and the docking structure, please refer to FIGS. 4-6D and the related descriptions, which will not be repeated here.

The one or more terminal devices 150 may include one or more terminal devices or software. In some embodiments, the one or more terminal devices 150 may include a mobile phone, a tablet, a laptop, a monitor, etc. In some embodiments, a user may view information and/or input data and/or instructions through the one or more terminal devices 150. In some embodiments, the one or more terminal devices 150 may include a signal transmitter and a signal receiver configured to communicate with the medical imaging system 140 to obtain and image relevant information of a subject.

In some embodiments, the one or more terminal devices 150 may be fixed and/or movable. For example, the one or more terminal devices 150 may be directly installed on the processor 110 and/or the medical imaging system 140, becoming part of the processor 110 and/or the medical imaging system 140. As another example, the one or more terminal devices 150 may be mobile device(s) that the user may carry to a location relatively far from the processor 110 and the medical imaging system 140. The one or more terminal devices 150 may connect and/or communicate with the processor 110 and the medical imaging system 140 via the signal transmission device 120.

It should be noted that the above description of the application scenario of the medical imaging system is merely for convenience of description and does not limit the present disclosure to the scope of the illustrated embodiments. It is understood that for those skilled in the art, after understanding the principles of the system, various components may be combined in any manner, or sub-components may be connected with other components without departing from these principles. In some embodiments, the processor and the storage device disclosed in FIG. 1 may be different units within one component, or one component may implement the functions of two or more components described above. For example, the components may share one memory unit, or each component may have its own storage unit. Such variations are all within the scope of protection of the present disclosure.

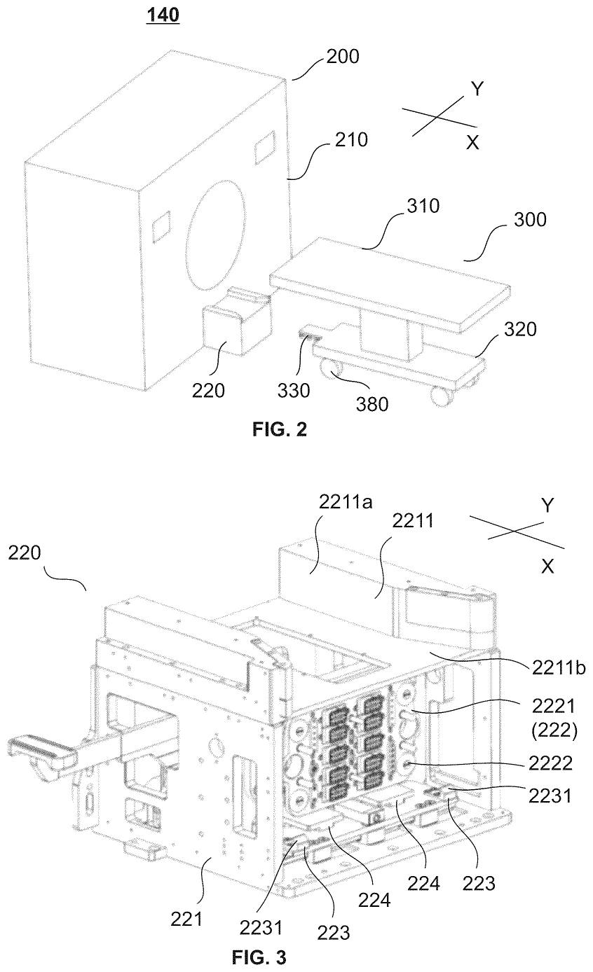

FIG. 2 is a schematic diagram of an exemplary structure of a medical imaging system according to some embodiments of the present disclosure.

As shown in FIG. 2, a medical imaging system 140 may include an imaging device 200 and a medical bed 300. The medical bed 300 is provided with a docking structure 330, and the imaging device 200 is provided with a connection structure 220. The docking structure 330 is configured to dock with the connection structure 220 to achieve the docking between the medical bed 300 and the imaging device 200.

As shown in FIG. 2, in some embodiments, the medical bed 300 includes a bed board 310, a supporting main body 320, and moving wheels 380. The bed board 310 is provided on a top of the supporting main body 320 to carry a patient. The moving wheels 380 are provided at a bottom of the supporting main body 320 to move the medical bed 300. The docking structure 330 is provided at an end of the supporting main body 320 to dock with the connection structure 220.

The imaging device 200 includes an imaging main body 210, and the connection structure 220 is set at one end of the imaging main body 210. The imaging main body 210 includes an imaging channel for imaging. In some embodiments, the bed board 310 is slidably connected with the supporting main body 320, so that the bed board 310 and the patient on the bed board 310 may move into the imaging channel of the imaging main body 210. By way of example, the imaging device 200 may be a computed tomography (CT) device, a positron emission tomography (PET) device, a magnetic resonance imaging (MRI) device, or the like, or a combination thereof (e.g., a PET-CT device, a PET-MR device, etc.).

In some embodiments, a direction in which the bed board 310 moves to move the patient into or out of the imaging channel of the imaging main body 210 is defined as a first direction X, and a direction perpendicular to the first direction X within a plane of the bed board 310 is defined as a second direction Y. The first direction is the direction along the length of the medical bed 300, i.e. the X direction shown in FIG. 2. The second direction is the direction along the width of the medical bed 300, i.e. the Y direction shown in FIG. 2. For example, the first direction X may be a longitudinal direction of the medical bed 300, and the second direction Y may be a lateral direction of the bed board 310. The docking structure 330 may be disposed at an end of the medical bed 300 (e.g., the supporting main body 320) in the first direction X facing the imaging device 200, and the connection structure 220 may be disposed at an end of the imaging device 200 (e.g., the imaging main body 210) in the first direction X facing the medical bed 300.

In some embodiments, the docking structure 330 may be detachably connected to the medical bed 300. Different medical beds 300 may be docked with corresponding imaging devices 200 through their respective docking structures 330, allowing the medical bed 300 to adapt to different imaging devices 200 and facilitating the maintenance and replacement of the docking structure 330.

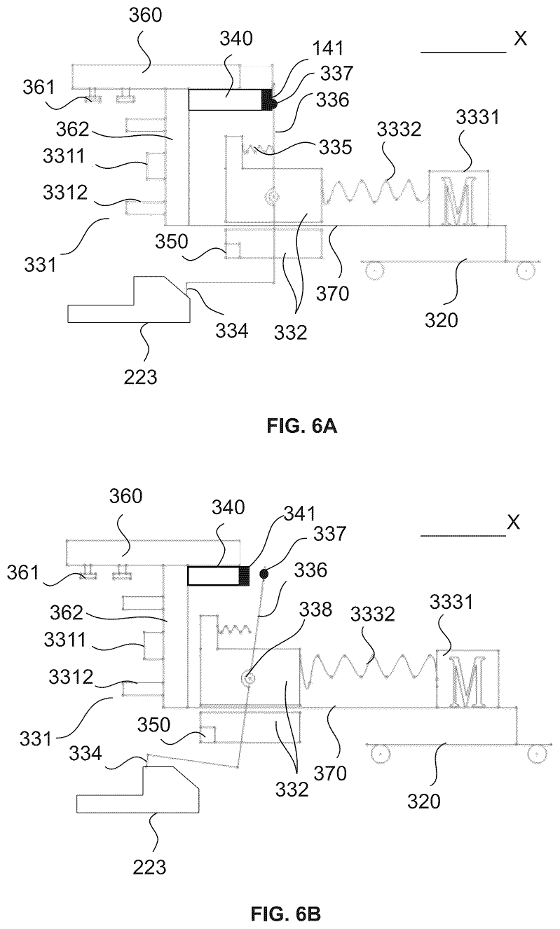

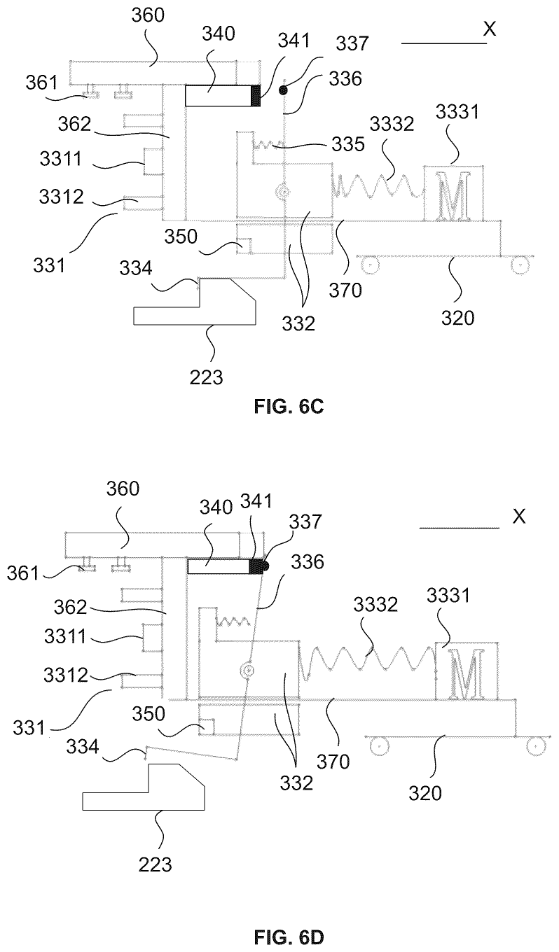

FIG. 3 is a schematic diagram of an exemplary structure of a connection structure according to some embodiments of the present disclosure. FIG. 4 is a schematic diagram of an exemplary structure of a docking structure according to some embodiments of the present disclosure. FIG. 5 is a schematic diagram of another perspective of the docking structure shown in FIG. 4. FIG. 6A is a schematic diagram of a medical bed in a first critical position according to some embodiments of the present disclosure. FIG. 6B is a schematic diagram of a medical bed in a second critical position according to some embodiments of the present disclosure. FIG. 6C is a schematic diagram of a medical bed in a third critical position according to some embodiments of the present disclosure. FIG. 6D is a schematic diagram of a separation of a docking structure and a connection structure according to some embodiments of the present disclosure.

Referring to FIGS. 3-6D, in some embodiments, the docking structure 330 comprises an electric driving assembly 333, the electric driving assembly 333 being provided on the medical bed 300, and the electric driving assembly 333 is at least configured to drive the medical bed 300 to move towards the imaging device 200, so as to achieving docking between the docking structure 330 and the connection structure 220, and achieving docking between the medical bed 300 and the imaging device 200. In some embodiments, the electric driving assembly 333 is also configured to drive the medical bed 300 to move horizontally or to move up and down.

In some embodiments, the connection structure 220 includes a docking base 221, an imaging-side connector 222, a stop portion 224, and a limiting part 223. The docking base 221 is arranged on the imaging main body 210, while the imaging-side connector 222, the limiting part 223, and the stop portion 224 are respectively arranged on the docking base 221.

The docking structure 330 includes a mounting seat 332. A tensioning part 334 is provided on the mounting seat 332. The tensioning part 334 has a locked position and an unlocked position, and the tensioning part 334 is configured to fit with the limiting part 223 of the connection structure 220 to lock or unlock a movement of the mounting seat 332. Specifically, when the tensioning part 334 is in the locked position and hooks with the limiting part 223 (as shown in FIG. 6C), the mounting seat 332 abuts against the stop portion 224. The hook between the tensioning part 334 and the limiting part 223 and the abutment between the mounting seat 332 and the stop portion 224 restricts the movement of the mounting seat 332 in a direction towards or away from the imaging device 220. In other words, the mounting seat 332 is fixedly connected to the connection structure 220, and the movement of the mounting seat 332 is locked. When the tensioning part 334 is in the unlocked position (e.g., as shown in FIGS. 6B and 6D) or when the tensioning part 334 is not hooked with the limiting part 223 (as shown in FIGS. 6A, 6B, and 6D), the mounting seat 332 is not fixedly connected to the connection structure 220, and the movement of the mounting seat 332 is unlocked.

The tensioning part 334 may include a hook structure. A side of the limiting part 223 that faces away from the imaging main body 210 has a slope 2231 inclined to the first direction X (as shown in FIG. 3). The slope 2231 may gradually rise from an end of the slope 2231 that is farther from the imaging main body 210 to an end that is closer to the imaging main body 210. The slope 2231 may be configured to abut against the tensioning part 134 to guide the tensioning part 134 to switch from the locked position to the unlocked position when the tensioning part 134 moves along the slope 2231. Specifically, when the tensioning part 134 abuts against the slope 2231 and moves towards the imaging main body 210, an abutting force exerted by the slope 2231 on the tensioning part 334 allows the tensioning part 334 to overcome an elastic force of the elastic part 335, thereby switching the tensioning part 334 from the locked position to the unlocked position. A side of the limiting part 223 that faces the imaging main body 210 may have a surface perpendicular to the first direction X (as shown in FIGS. 6A-6D). The cooperation of the hook structure with the surface perpendicular to the first direction X achieves the hook between the tensioning part 334 and the limiting part 223.

In some embodiments, an elastic part 335 may be provided between the tensioning part 334 and the mounting seat 332. The elastic part 335 may be configured to restore the tensioning part 334 from the unlocked position to the locked position. For example, the elastic part 335 may restore the tensioning part 334 from the unlocked position shown in FIG. 6B to the locked position shown in FIG. 6C.

In some embodiments, the docking structure 330 may further include a bed-side connector 331. The bed-side connector 331 may be provided at one end of the medical bed 300 that docks with the imaging device 200. The electric driving assembly 333 may be drivingly connected to the mounting seat 332. The electric driving assembly 333 may be at least configured such that when the tensioning part 334 is in the locked position and hooks with the limiting part 223 of the connection structure 220, the electric driving assembly 333 drives the medical bed 300 to move towards the mounting seat 332, so as to cause the medical bed 300 to move relative to the mounting seat 332, thereby achieving docking between the bed-side connector 310 and the connection structure 220, and achieving docking between the medical bed 300 and the imaging device 200. Specifically, as shown in FIG. 6C, when the tensioning part 334 is in the locked position and hooks with the limiting part 223, the mounting seat 332 abuts against the stop portion 224, and the mounting seat 332 is fixedly connected to the connection structure 220, thereby preventing the mounting seat 332 from moving in a direction (i.e., the first direction X shown in FIG. 6C) towards or away from the connection structure 220. At this time, when the electric driving assembly 333 operates, a distance between the medical bed 300 and the mounting seat 332 is reduced, causing the medical bed 300 to move in a direction (i.e., a left direction of the first direction X shown in FIG. 6C) towards the mounting seat 332, thereby bringing the medical bed 300 closer to the imaging device 200, and docking the bed-side connector 310 with the connection structure 220.

By providing the electric driving assembly 333 on the medical bed 300, when docking the medical bed 300 docks with the imaging device 200, a driving source 3331 (e.g., a motor) of the electric driving assembly 333 is relatively far from a magnetic field of a magnet (not shown in the drawings) of the imaging device 200, which minimizes an interference between the driving source 3331 (e.g., the motor) and the magnetic field of the imaging device 200, allowing the driving source 3331 (e.g., the motor) to be positioned without the need for alignment parallel to a direction of the magnetic field. The structural flexibility is improved. Additionally, extra field-shaping devices may not be needed for the magnet of the imaging device 200, thereby reducing structural complexity and material costs. Moreover, using the electric driving assembly 333 also improves the reliability of docking between the medical bed 300 and the imaging device 200, thereby enhancing operational comfort.

In some embodiments, the bed-side connector 331 may further include a height adjustment mechanism. The height adjustment mechanism may be configured to adjust a height of the bed-side connector 331 relative to the connection structure 220. That is, the height adjustment mechanism may adjust a height of the bed-side connector 331 relative to the ground such that the bed-side connector 331 is at a height adapted to a height of the connection structure 220 even on uneven ground, thereby reducing the difficulty of docking and improves docking accuracy.

By way of example, the height adjustment mechanism may include a slide rail and a slider arranged along a height direction of the height adjustment mechanism. The slide rail may be fixed to the medical bed 300, and the bed-side connector 331 may be connected to the slider. The slider may be provided with a positioning pin. When the positioning pin is loosened, the slider may move within the slide rail, thereby adjusting the height of the bed-side connector 331. When the bed-side connector 331 reaches an appropriate height, the positioning pin may be tightened to compress the slider and the slide rail together, thereby restricting a movement of the slider and maintaining the bed-side connector 331 at the appropriate height. In some embodiments, a vertical movement of the slider within the slide rail may be controlled by a motor. The motor may be connected to the processor 110 via a signal, allowing the processor 110 to control the height of the bed-side connector 331, thereby improving precision.

The electric driving assembly 333 may include the driving source 3331 and a transmission assembly 3332. The driving source 3331 may be fixedly connected to the medical bed 300 (e.g., the supporting main body 320). An input end of the transmission assembly 3332 may be connected to the driving source 3331, and an output end of the transmission assembly 3332 may be connected to the mounting seat 332. The transmission assembly 3332 may be configured to convert rotational motion output by the driving source 3331 into linear motion, so that when an output shaft of the driving source 3331 rotates, the transmission assembly 3332 can enable the mounting base 332 and the medical bed 300 (for example, the support body 320) to move relative to each other along the first direction X. When the tensioning part 334 is in the locked position and hooks with the limiting part 223 (as shown in FIG. 6C), the movement of the mounting seat 332 is locked. In this case, when the driving source 3331 is working, the transmission assembly 3332 pulls the driving source 3331 along the first direction X toward the mounting seat 332, that is, pulls the medical bed 300 along the first direction X toward the mounting seat 332.

In some embodiments, the driving source 3331 may include a drive motor. In some embodiments, the transmission assembly 3332 may include a lead screw nut mechanism, a crank slider mechanism, etc. For example, when the transmission assembly 3332 includes a lead screw nut mechanism, a lead screw is connected to the output shaft of the driving source 3331, and a nut is threadedly engaged with the lead screw. The nut is fixedly connected to the mounting seat 332.

In some embodiments, when the driving source 3331 is powered off, the transmission assembly 3332 may move freely. Thus, the medical bed 300 (e.g., the supporting main body 320) may be manually pushed towards the imaging main body 210. Through the motion of the transmission assembly 3332, it is possible to move the bed-side connector 331 towards the imaging-side connector 222 when the mounting seat 332 remains stationary. Specifically, when the transmission assembly 3332 is a lead screw nut mechanism, a lead screw may be connected to the output shaft of the motor, and a nut may be threadedly matched with the lead screw. The nut may be fixedly connected to the mounting seat 332. When the driving source 3331 is powered off, the lead screw may freely rotate relative to the output shaft of the motor. When the medical bed 300 reaches a third critical position, the limiting part 223 may block the tensioning part 334 from moving in the first direction X away from the imaging main body 210, thereby preventing the mounting seat 332 from moving in the first direction X away from the imaging main body 210. At this time, if the mounting seat 332 and the nut are stationary, manually pushing the medical bed 300 (e.g., the supporting main body 320) towards the imaging main body 210 may cause the lead screw to rotate relative to the nut and move along the first direction X, which enables the medical bed 300 (e.g., the supporting main body 320) to drive the bed-side connector 331 to move towards the imaging-side connector 222 for docking, thereby achieving manual docking.

In some embodiments, the medical bed 300 may include a guide rail 370 arranged on the supporting main body 320. The mounting seat 332 may be configured to move along the guide rail 370 in the first direction X. The guide rail 370 may guide a relative movement between the mounting seat 332 and the medical bed 300 (e.g., the supporting main body 320) in the first direction X.

In some embodiments, when the medical bed 300 and the imaging device 200 are in a docked state, the driving source 3331 is arranged at a position where a magnetic field strength of a magnet of the imaging device 200 is 5 mT-200 mT. By placing the drive source 3331 at a location with low magnetic field strength, interference between the driving source 3331 and the magnetic field of the magnet may be reduced.

In some embodiments, in the docked state, a distance between the driving source 3331 and the side of the magnet facing the medical bed 300 may be positively correlated with a maximum magnetic field strength of the magnet. The distance refers to a shortest distance between a side of the driving source 3331 facing the imaging device 200 and a side of the magnet facing the medical bed 300. The maximum magnetic field strength of the magnet refers to the magnetic field strength at the center of the magnet. When the maximum magnetic field strength of the magnet is relatively low, the interference between the driving source 3331 and the magnetic field is relatively weak, and the distance between the driving source 3331 and the magnet may be relatively close. When the maximum magnetic field strength of the magnet is relatively high, the interference between the driving source 3331 and the magnetic field is relatively strong, and the distance between the driving source 3331 and the magnet may be relatively far. By designing the distance between the driving source 3331 and the magnet, interference between the driving source 3331 and the magnetic field of the magnet may be reduced, the driving source 3331 may be prevented from being too far from the magnet, which may cause the driving source 3331 to extend deep into an interior of the medical bed 300, thereby saving an internal space of the medical bed 300.

In some embodiments, in a docked state, when the maximum magnetic field strength of the magnet is 1.5 T, the distance between the driving source 3331 and the magnet of the imaging device 200 is within a range from 20 cm to 40 cm. In some embodiments, in a docked state, when the maximum magnetic field strength of the magnet is 3 T, the distance between the driving source 3331 and the magnet of the imaging device 200 is within a range from 40 cm to 60 cm. For example, when the maximum magnetic field strength of the magnet is 3 T and the driving source 3331 is arranged at a position where the magnetic field strength of the magnet of the imaging device 200 is 200 mT, the distance between the driving source 3331 and the magnet in the docked state may be 45 cm.

In some embodiments, the electric driving assembly 333 may further include a moving mechanism (not shown in the drawings). The driving source 3331 may be arranged on the moving mechanism. The moving mechanism may be configured to adjust a position of the driving source 3331, thereby the driving source 3331 is arranged at positions corresponding to different magnetic field strengths of the magnet of the imaging device in the docking state, so that the driving source 3331 is arranged at a position with low magnetic field strength. On the other hand, the moving mechanism can adjust a position of the driving source 3331, thereby adjusting the distance between the driving source 3331 and the side of the magnet facing the medical bed 300 in the docked state, so that the docking structure 330 may adapt to the magnet of the imaging device 200 with different maximum magnetic field strengths.

As shown in FIGS. 4-6D, in some embodiments, the docking structure 330 includes a tensioning assembly. The tensioning assembly includes a rotating shaft 338 set on the mounting seat 332 and a pull rod 336 rotatably connected to the rotating shaft 338. One end of the pull rod 336 is provided with the tensioning part 334. The pull rod 336 rotates around the rotating shaft 338, thereby driving the tensioning part 334 to switch between the locked position and the unlocked position.

In some embodiments, the elastic part 335 may include a helical spring, an elastic block, etc. One end of the elastic part 335 may be fixed to the mounting seat 332, and another end of the elastic part 335 may be fixed to the pull rod 336. In some embodiments, the elastic part 335 may further include a torsion spring. The torsion spring may be sleeved on the rotating shaft 338.

In some embodiments, the docking structure 330 may further include a supporting part 340. The supporting part 340 may be connected to the medical bed 300 (e.g., the supporting main body 320). The bed-side connector 331 may be set on the supporting part 340 (as shown in FIG. 4), and the supporting part 340 may provide a platform on the medical bed 300 for installing the bed-side connector 331, thereby allowing the bed-side connector 331 to move synchronously with the medical bed 300.

In some embodiments, the supporting part 340 may include a first side plate 340a and a second side plate 340b spaced apart along the second direction Y, as shown in FIG. 5. The mounting seat 332 may be located between the first side plate 340a and the second side plate 340b. Two ends of the bed-side connector 331 may be fixedly connected to the first side plate 340a and the second side plate 340b, respectively.

In some embodiments, the first side plate 340a and the second side plate 340b of the supporting part 340 may be provided with avoidance holes 342, as shown in FIG. 4. The rotating shaft 338 may pass through the mounting seat 332, and two ends of the rotating shaft 338 may extend out of the avoidance holes 342 on two sides of the supporting part 340. The pull rod 336 may be connected to an end of the rotating shaft 338 protruding from the avoidance holes 342, and may be rotatably connected to the mounting seat 332 through the rotating shaft 338.

In some embodiments, the avoidance holes 342 may extend in the first direction X, thereby allowing the rotating shaft 338 to move along the first direction X within the avoidance holes 342. Thus, the mounting seat 332 is permitted to move relative to the medical bed 300 (e.g., the supporting main body 320) along the first direction X.

In some embodiments, the supporting part 340 may be provided with a stopping part 341, as shown in FIG. 4. A projection of the stopping part 341 may at least partially overlaps with an end of the pull rod 336 away from the tensioning part 334 in the first direction X, as shown in FIGS. 6A to 6D. The stopping part 341 may be configured to separate the medical bed 300 from the imaging device 200. Specifically, when the medical bed 300 is separated from the docking state as shown in FIG. 6C, the electric driving assembly 333 drives the medical bed 300 away from the mounting seat 332, and the supporting part 340 synchronously moves in a direction (i.e., the right direction of the first direction X as shown in FIGS. 6A to 6D) away from the imaging device 200. When the stopping part 341 contacts the end of the pull rod 336 away from the tensioning part 334, the stopping part 341 moves. The pull rod 336 rotates around the rotating shaft 338, thereby switching the tensioning part 334 from the locked position to the unlocked position, and the elastic part 335 deforms, as shown in FIG. 6D. At this point, the movement of the mounting seat 332 is unlocked, and the mounting seat 332 moves together with the medical bed 300, thereby separating the medical bed 300 from the imaging device 200.

Please refer to FIGS. 4 and 5. In some embodiments, the tensioning assembly may include two pull rods 336 arranged at interval in the second direction Y, as shown in FIGS. 4 and 5. The two pull rods 336 may be rotatably connected to the rotating shaft 338. The two pull rods 336 may be provided with a tensioning part 334, respectively. Each of the two tensioning parts 334 cooperates with a corresponding limiting part 223. Each end of the two pull rods 336 away from the corresponding tensioning part 334 is connected by a connecting rod 337. As shown in FIGS. 6A to 6D, a projection of the stopping part 341 at least partially overlaps with the connecting rod 337 in the first direction X. When the medical bed 300 and the imaging device 200 separate from the docking state as shown in FIG. 6C, the stopping part 341 may contact the connecting rod 337, thereby simultaneously causing the two pull rods 336 to rotate, switching the two tensioning parts 334 from the locked position to the unlocked position.

In some embodiments, the docking structure 330 may further include a guiding part 360, which may be connected to the bed-side connector 331. The guiding part 360 may be configured to fit with the guidance part 2211 of the connection structure 220 to guide the docking of the bed-side connector 331 with the imaging-side connector 222 of the connection structure 220. Specifically, the guidance part 2211 may guide the guiding part 360 to move along the first direction X, enabling the medical bed 300 (e.g., the supporting main body 320) to move accurately along the first direction X, thereby enhancing the docking precision of the bed-side connector 331 with the imaging-side connector 222 and reducing the difficulty of docking.

Please refer to FIG. 3. In some embodiments, the guidance part 2211 may include a connected precision guidance segment 2211a and a coarse guidance segment 2211b. The coarse guidance segment 2211b may be positioned at an entrance of the precision guidance segment 2211a, and a width of the coarse guidance segment 2211b may gradually increase in a direction away from the precision guidance segment 2211a, facilitating the entry of the guiding part 360 from the coarse guidance segment 2211b into the guidance part 2211. Due to the uniform width of the precision guidance segment 2211a, the guiding part 360 may accurately move along the first direction X under the guidance of the precision guidance segment 2211a when transitioning from the coarse guidance segment 2211b to the precision guidance segment 2211a.

Please refer to FIG. 4. In some embodiments, the guiding part 360 may be mounted on the medical bed 300 (e.g., the supporting main body 320) via an installation plate 362. The guiding part 360 may include guiding pulleys 361 that slide along the guidance part 2211, facilitating the movement of the guiding part 360 along the guidance part 2211. In some embodiments, the guiding pulleys 361 may be positioned on two sides of the guiding part 360 along the second direction Y. A distance between outer end faces of the guiding pulleys 361 on the two sides of the guiding part 360 along the second direction Y matches the width of the precision guidance segment 2211a, allowing the guiding part 360 to be accurately guided to move along the first direction X when the guiding pulleys 361 on the two sides of the guiding part 360 along the second direction Y cooperate with the precision guidance segment 2211a.

In some embodiments, one of the bed-side connector 331 and the imaging-side connector 222 is a male-end connector, and the other is a female-end connector. For example, the bed-side connector 331 may be a male-end connector, and the imaging-side connector 222 may be a female-end connector. The female-end connector may include a socket 2221, and the male-end connector may include a pin 3311. The pin 3311 and the socket 2221 cooperate for insertion and withdrawal. In some embodiments, the socket 2221 may be provided with a guiding sleeve 2222, and the male-end connector further includes a guiding pin 3312. The guiding sleeve 2222 and the guiding pin 3312 may be inserted and coordinated to guide a docking direction between the male-end connector and the female-end connector.

Please refer to FIG. 6A. In some embodiments, when the medical bed 300 moves towards the imaging device 200 and the tensioning part 334 has not yet contacted the limiting part 223, the tensioning part 334 remains in the locked position, and the elastic part 335 is in its natural state. A relative position of the mounting seat 332 with respect to the medical bed 300 (including the bed-side connector 331, supporting part 340, and stopping part 341) remains unchanged. When the tensioning part 334 just contacts with the limiting part 223, the medical bed 300 is at the first critical position as shown in FIG. 6A.

The medical bed 300 continues to move towards the imaging device 200 from the first critical position. Under an action of the inclined slope 2231, the tensioning part 334 gradually moves along the inclined slope 2231, transitioning from the locked position to the unlocked position, and the elastic part 335 stretches and undergoes elastic deformation. When the tensioning part 334 is about to pass over the limiting part 223 but has not yet passed it, the elastic part 335 reaches its maximum stretch, the tensioning part is in the unlocked position, and the medical bed 300 is at the second critical position as shown in FIG. 6B.

The medical bed 300 continues to move towards the imaging device 200 from the second critical position. The tensioning part 334 passes over the inclined slope 2231 and hooks with a flat surface of the limiting part 223, cooperating with the abutment of the mounting seat 332 with the stop portion 224, thereby securely connecting the mounting seat 332 with the connection structure 220. At this point, the elastic part 335 is restored to its original shape, and the tensioning part 334 transitions from the unlocked position shown in FIG. 3 to the locked position shown in FIG. 6C under the action of the elastic part 335. The medical bed 300 is now at the third critical position as shown in FIG. 6C.

After the medical bed 300 reaches the third critical position, the electric driving assembly 330 may be initiated. Since the movement of the mounting seat 332 is restricted, the electric driving assembly 330 drives the medical bed 300 (or the supporting main body 320) to move closer to the mounting seat 332 (or the connection structure 220) until the docking structure 330 is docked with the connection structure 220.

In some embodiments, when the medical bed 300 and the imaging device 200 separate from the docking state, the medical bed 300 moves away from the imaging device 200 from the third critical position. When the stopping part 341 contacts the end of the pull rod 336 (or the connecting rod 337) away from the tensioning part 334, the stopping part 341 moves, causing the pull rod 336 to rotate about the axis 338, thereby switching the tensioning part 334 from the locked position to the unlocked position, and causing elastic deformation of the elastic part 335 as shown in FIG. 6D. At this point, the movement of the mounting seat 332 is unlocked, and the mounting seat 332 moves together with the medical bed 300, thus separating the medical bed 300 from the imaging device 200.

In some embodiments, the docking structure 330 may further include a signal generator 350. The signal generator 350 may be provided on the tensioning part 334. When the tensioning part 334 hooks with the limiting part 223 (i.e., when the tensioning part 334 contacts the flat surface of the limiting part 223), the medical bed 300 is in the third critical position, and the signal generator 350 may output a sensing signal.

In some embodiments, as shown in FIGS. 6A to 6D, the signal generator 350 may be connected to a front end of the mounting seat 332. When the mounting seat 332 abuts against the stop portion 224, the medical bed 300 is in the third critical position, and the signal generator 350 may output the sensing signal. The signal generator 150 may generate the sensing signal.

The sensing signal represents that the medical bed 300 is in the third critical position. The sensing signal is configured to determine a moment when the medical bed 300 reaches the third critical position. In response to receiving the sensing signal, the processor 110 may directly control the electric driving assembly 333 to initiate, or output corresponding prompt information (e.g., in forms of light signals, graphical information, sound signals, etc.) to prompt the user to manually control the electric driving assembly 333 to initiate.

In some embodiments, the docking structure 330 and the medical bed 300 may be detachably connected. In an installed state, a relative position of the bed-side connector 331 with respect to the medical bed 300 may be fixed, a relative position of the driving source 3331 with respect to the medical bed 300 may be fixed, and a relative position of the mounting seat 332 with respect to the driving source 3331 may be variable. The electric driving assembly 3331 moves the bed-side connector 331 and the medical bed 300 (e.g., the supporting main body 320) relative to the mounting seat 332, such that the medical bed 300 is close to the imaging device 200 and docks with the imaging device 200.

In some embodiments, the docking structure 330 may be provided with a sensor (not shown in the drawings). The sensor may be configured to measure an orientation of the docking structure 330 relative to the connection structure 220 of the imaging device 200, thereby improving the docking accuracy and reducing the difficulty of docking. In some embodiments, the orientation may include a direction and/or a distance of the docking structure 330 relative to the connection structure 220 of the imaging device 200. In some embodiments, the sensor may be provided on the medical bed 300.

In some embodiments, the sensor may include one or more cameras that determine a direction and a distance between the docking structure 330 and the connection structure 220 through image analysis. In some embodiments, the sensor may include a plurality of cameras. The plurality of cameras may be calibrated with each other to enhance the measurement accuracy.

In some embodiments, the sensor may further include a distance sensor. For example, the sensor may include an ultrasonic receiver, and an ultrasonic generator may be provided at a position on the imaging device 200 or near the imaging device 200. The ultrasonic receiver may determine the orientation of the docking structure 330 relative to the connection structure 220 of the imaging device 200 based on the position of the ultrasonic generator and an ultrasonic signal emitted or reflected by the imaging device 200. As another example, the sensor may include a laser receiver, and a laser generator may be provided at a position on the imaging device 200 or near the imaging device 200. The laser receiver may determine the orientation of the docking structure 330 relative to the connection structure 220 of the imaging device 200 based on the position of the laser generator and a laser signal emitted or reflected by the imaging device 200.

In some embodiments, markers may be arranged on the imaging device 200 and the surrounding ground. The sensor may measure related markers to determine the orientation of the docking structure 330 relative to the connection structure 220 of the imaging device 200. In some embodiments, based on measurements from the sensor and the related markers, real-time control of a movement parameter (e.g., a moving direction, a path of movement, a moving speed, etc.) of the medical bed 300 may be performed.

In some embodiments, a display device (not shown in the drawings) may be installed on the medical bed 300. The display device may perform as one of the one or more terminal devices 150, and connect to the sensor via a signal. The display device may be configured to display a measurement result of the sensor.

In some embodiments, the processor 110 may automatically adjust the movement parameter of the medical bed 300 based on the measurement result of the sensor. In some embodiments, the processor 110 may issue prompt information (e.g., in forms of sound signals, light signals, etc.) based on the measurement result of the sensor to prompt the user to manually adjust the movement parameter of the medical bed 300. The movement parameter of the medical bed 300 may include at least one of a moving direction, a moving speed, or a height of the medical bed 300 relative to the ground.

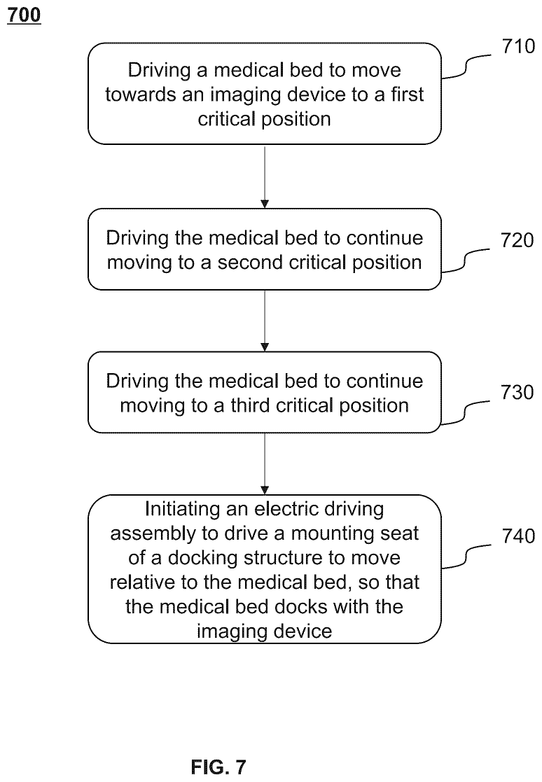

FIG. 7 is a flowchart of an exemplary docking process between a medical bed and an imaging device according to some embodiments of the present disclosure. In some embodiments, process 700 may be executed by the medical imaging system 140 or the processor 110. For example, process 700 may be implemented as a set of instructions stored in a storage device (e.g., the storage device 130) inside or outside the processor 110, and accessible by the processor 110. The processor 110 may execute the set of instructions and, when executing the set of instructions, the processor 110 may be configured to perform process 700. The operations described in process 700 are intended for illustrative purposes. In some embodiments, additional operations not described herein and/or one or more operations not discussed may be added to complete the process.

In some embodiments, process 700 may include operations 710-740.

In 710, the medical bed 300 may be driven to move towards the imaging device 200 to a first critical position.

In some embodiments, the first critical position refers to a position of the medical bed 300 when the tensioning part 334 just contacts the limiting part 223, as shown in FIG. 6A. At this time, the tensioning part 334 is in a locked position, and the elastic part 335 is in a natural state.

In some embodiments, the docking structure 330 and the medical bed 300 may be detachably connected. Prior to operation 710, process 700 may also include: connecting the docking structure 330 to the medical bed 300 to achieve docking between the docking structure 330 and the connection structure 220, thereby docking the medical bed 300 with the imaging device 200.

In some embodiments, at least one of the docking structure 330 or the medical bed 300 may be provided with a sensor that may measure an orientation of the docking structure 330 relative to the connection structure 220.

In some embodiments, operation 710 may include: adjusting a movement parameter of the medical bed 300 based on a measurement result of the sensor.

In some embodiments, the processor 110 may automatically adjust the movement parameter of the medical bed 300 based on the measurement result of the sensor. In some embodiments, the processor 110 may issue prompt information (e.g., in forms of sound signals, light signals, etc.) based on the measurement result of the sensor to prompt a user to manually adjust the movement parameter of the medical bed 300.

In some embodiments, the movement parameter of the medical bed 300 include at least one of a moving direction, a moving speed, or a height of the medical bed 300 relative to the ground. Adjusting the moving direction may enable the medical bed 300 to orient towards the imaging device 200, thereby enhancing docking accuracy. Adjusting the moving speed may enable the medical bed 300 to approach the imaging device 200 quickly or smoothly dock with the imaging device 200, thereby improving docking efficiency and safety. Adjusting the height of the medical bed 300 may place the docking structure 330 at a height compatible with the connection structure 220, thereby enhancing docking accuracy and reducing docking difficulty.

In some embodiments, operation 710 may further include: controlling, by the processor 110, based on the measurement result of the sensor, a height adjustment mechanism to adjust a height of the bed-side connector 331. Adjusting the height of the bed-side connector 331 may place the bed-side connector 331 at a height compatible with the imaging-side connector 222, thereby enhancing docking accuracy and reducing docking difficulty.

In some embodiments, the processor 110 may automatically control the height adjustment mechanism to adjust the height of the bed-side connector 331 based on the measurement result of the sensor. In some embodiments, the processor 110 may issue prompt information (e.g., in forms of sound signals, light signals, etc.) based on the measurement result of the sensor to prompt the user to manually adjust the height adjustment mechanism to adjust the height of the bed-side connector 331.

In 720, the medical bed 300 may be driven to continue moving to a second critical position.

In some embodiments, the second critical position refers to a position of the medical bed 300 when the tensioning part 334 is about to move across but has not yet moved across the limiting part 223, as shown in FIG. 6B. At this time, the tensioning part 334 is in the unlocked position, and the elastic part 335 is stretched to a maximum extent.

During the process of the medical bed 300 moving from the first critical position to the second critical position, the tensioning part 334 gradually moves along the slope 2231 under an action of the slope 2231. The locked position is transited to the unlocked position, and the elastic part 335 stretches and undergoes elastic deformation.

In 730, the medical bed 300 may be driven to continue moving to a third critical position.

In some embodiments, the third critical position refers to a position of the medical bed 300 when the tensioning part 334 has just moved across the limiting part 223, as shown in FIG. 6C. At this time, the tensioning part 334 is restored from the unlocked position to the locked position under an action of the elastic part 335. The tensioning part 334 hooks with the limiting part 223, and cooperate with the abutment between the mounting seat 332 and the stop portion 224, thereby connecting and relatively fixing the mounting seat 332 with the connection structure 220.

In 740, the electric driving assembly 333 may be initiated to drive the mounting seat 332 to move relative to the medical bed 300, so that the medical bed 300 docks with the imaging device 200.

In some embodiments, the tensioning part 334 may be provided with a signal generator 350. The signal generator 350 may be configured to output a sensing signal when the tensioning part 334 hooks with the limiting part 223 (e.g., when the tensioning part 334 contacts a flat surface of the limiting part 223 directly). The sensing signal may indicate that the medical bed 300 is at the third critical position, thereby facilitating a determination of a moment when the medical bed 300 reaches the third critical position.

Operation 740 may include: in response to receiving the sensing signal, initiating the electric driving assembly 333. In some embodiments, in response to receiving the sensing signal, the processor 110 may directly control the electric driving assembly 333 to initiate, or output corresponding prompt information (e.g., in forms of light signals, graphical information, sound signals, etc.) to prompt the user to manually control the electric driving assembly 333 to initiate.

At the third critical position, the tensioning part 334 hooks with the limiting part 223, and the mounting seat 332 abuts with the stop portion 224, thereby locking the movement of the mounting seat 332. After initiating the electric driving assembly 333, the electric driving assembly 333 drives the mounting seat 332 in a direction away from the imaging device 200, so that a driving force acting on the mounting seat 332 reacts against the medical bed 300, causing the medical bed 300 to move towards the imaging device 200 to complete the docking.

It should be noted that any of operations 710, 720, 730, and 740 may be automatically implemented by the processor 110 (for example, in operations 710, 720, 730, the processor 110 may control a driving motor of the moving wheels 380 of the medical bed 300 to move the medical bed 300), or implemented manually through user operation (for example, in operations 710, 720, 730, the user may manually push the medical bed 300 to move).

In practical scenarios, due to unforeseen circumstances such as power loss, the medical bed 300 may experience difficulty in movement. For example, the power loss prevents the stopping part 341 from driving the tensioning part 334 to switch to the unlocked position, such that the limiting part 223 remains obstructing the tensioning part 334 in the locked position, thereby preventing the medical bed 300 from moving in a direction away from the imaging main body 210, and thus the bed-side connector 331 is unable to be detached from the imaging-side connector 222. Some embodiments of the present disclosure provide a docking detachment structure 400 that coordinates with the docking structure 330 described above. The docking detachment structure 400 may be provided on the connection structure 220 to achieve mechanical detachment of the medical bed 300 from the imaging device 200. The docking detachment structure 400 prevents the medical bed 300 from being locked to the imaging device 200, thereby meeting safety standards required by the medical industry.

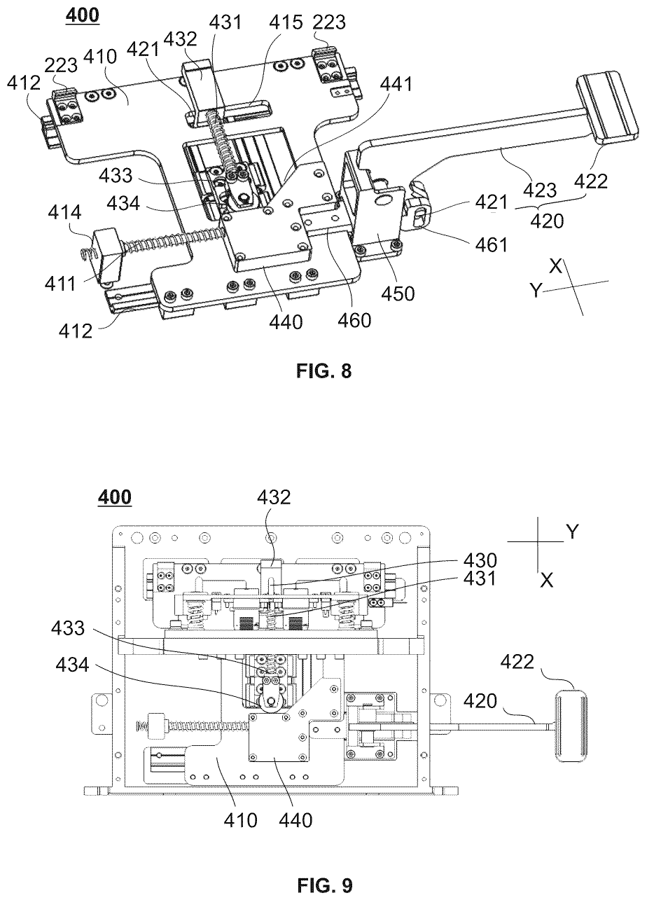

FIG. 8 is a schematic diagram of an exemplary structure of a connection structure according to some embodiments of the present disclosure. FIG. 9 is a schematic diagram of another perspective of the connection structure shown in FIG. 8 according to some embodiments of the present disclosure.

Referring to FIGS. 3, 8, and 9, some embodiments of the present disclosure further provide a docking detachment structure 400. The docking detachment structure 400 is configured such that: under an action of external force, the docking detachment structure 400 causes the docking structure 330 and the connection structure 220 to separate along the first direction X. And a direction of the external force is perpendicular to the first direction X, that is the external force is located in a plane perpendicular to the first direction X.

The docking detachment structure 400 may include: a docking base 221, a sliding part 410, a first elastic member 411, and an operating part 420. The docking base 221 may be provided on the imaging main body 210 and located on a side of the imaging main body 210 along a first direction X. The first direction X is axial along an imaging channel of the imaging main body 210. The sliding part 410 may be slidably connected to the docking base 221 along a second direction Y. The sliding part 410 may be provided with a limiting part 223, and the sliding part 410 may have a blocking position and a releasing position along the second direction Y relative to the docking base 221. In the blocking position, the limiting part 223 may block the movement of the tensioning part 334 of the medical bed 300 along the first direction X away from the imaging main body 210. In the releasing position, the limiting part 223 may release the blocking of the tensioning part 334. The docking base 221 and the sliding part 410 may be connected to the first elastic member 411 respectively. The first elastic member 411 may be configured to drive the sliding part 410 from the releasing position to the blocking position. The operating part 420 may be rotationally connected to the docking base 221 about an axis of the first direction X. When the operating part 420 rotates relative to the docking base 221 under the action of external force, the operating part 420 may switch the sliding part 410 from the blocking position to the releasing position along the second direction Y, so as to allow the docking structure 330 to separate from the connection structure 220 along the first direction X.

In some embodiments, the operating part 420 includes an operating rod 423 and an actuating part 422. An end of the operating rod 423 away from the docking base 221 may be connected to the actuating part 422. The actuating part 422 may include a foot pedal, allowing the user to rotate the operating part 420 by stepping on the foot pedal, which facilitates operation.

In some embodiments, the docking detachment structure 400 may further include an ejector part 430 and a second elastic member 431. The ejector part 430 may be slidably connected to the docking base 221 along the first direction X. The ejector part 430 and the docking base 221 may be connected to the second elastic member 431, respectively. The ejector part 430 may have an eject position and a return position along the first direction X. In the return position, the ejector part 430 may allow the medical bed 300 (e.g., the supporting main body 320) to drive the bed-side connector 331 to dock with the imaging-side connector 222. In the eject position, the ejector part 430 may push the medical bed 300 (e.g., the supporting main body 320) to detach the bed-side connector 331 from the imaging-side connector 222. The second elastic member 431 may be configured to drive the ejector part 430 to switch from the eject position to the return position.

In some embodiments, the sliding part 410 may be provided with a linkage part 440. After the sliding part 410 switches to the releasing position, when the operating part 420 rotates relative to the docking base 221, the operating part may push the sliding part 410 to move along the second direction Y, thereby causing the sliding part 410 to drive the linkage part 440 to push the ejector part 430 from the return position to the eject position.

In some embodiments, the linkage part 440 may be positioned on a side of the ejector part 430 closer to the imaging main body 210. The linkage part 440 may have a linkage surface 441 facing the ejector part 430 for abutting with the ejector part 430. The linkage surface 441 may be inclined relative to the first direction X and the second direction Y. From an end of the linkage part 440 near the medical bed 300 to an end of the linkage part 440 away from the medical bed 300, the linkage part 440 may incline towards a direction from the releasing position to the blocking position, thereby allowing the linkage part 440 to push the ejector part 430 to switch from the return position to the eject position through the linkage surface 441.

In some embodiments, the ejector part 430 may be provided with a roller 434 at an end of the ejector part 430 away from the medical bed 300. The roller 434 may roll against the linkage surface 441. By rolling the roller 434 against the linkage surface 441, sliding friction during a movement of the ejector part 430 is converted into rolling friction, thereby reducing resistance when pushing the ejector part 430 to the eject position.

In some embodiments, an ejector rail 432 may be provided on the docking base 221. The ejector part 430 may move along the first direction X in cooperation with the ejector rail 432, thereby accurately guiding a moving direction of the ejector part 430, facilitating accurate switching of the ejector part 430 between the eject position and the return position. An end of the ejector part 430 away from the medical bed 300 may extend to the ejector rail 432 and cooperate with the linkage part 440.

In some embodiments, a protruding part 433 may be provided on the end of the ejector part 430 away from the medical bed 300. The second elastic member 431 may be mounted on the ejector part 430 and located between the protruding part 433 and the ejector rail 432. One end of the second elastic member 431 may abut against the protruding part 433, and the other end of the second elastic member 431 may abut against the ejector rail 432. Thus, the second elastic member 431 provides resilience between the ejector part 430 and the docking base 221 through the ejector rail 432 and the protruding part 433.

In some embodiments, the roller 434 may be installed on the protruding part 433. For example, the protruding part 433 may be configured as a mounting bracket for the roller 434. Alternatively, an additional mounting bracket may be added on the protruding part 433 for installing the roller 434.

In some embodiments, the sliding part 410 is slidably connected to the docking base 221 along the second direction Y via a slide rail 412. A slider may be fixedly provided on a side of the sliding part 410 facing the slide rail 412. The slider may be slidably connected with the slide rail 412 along the second direction Y.

In some embodiments, the sliding part 410 may be provided with an avoidance groove 415, and a connecting part 4321 may be provided at an end of the ejector rail 432 away from the medical bed 300. The connecting part 4321 may pass through the avoidance groove 415 and be fixedly connected to the docking base 221. The connecting part 4321 may move in cooperation with the avoidance groove 415 along the second direction Y. Thus, when the sliding part 410 moves along the second direction Y, the connecting part 4321 moves relative to the avoidance groove 415, thereby connecting the ejector rail 432 with the docking base 221, and allowing the sliding part 410 to move along the second direction Y relative to the docking base 221 and the ejector rail 432.

In some embodiments, the sliding part 410 may include a slide plate, and the limiting part 223 and the linkage part 440 may be set on the slide plate.

In some embodiments, one of the operating part 420 and the sliding part 410 may be provided with a slide shaft 421, and the other may be provided with a slide groove 461. The slide shaft 421 slidably cooperates with the slide groove 461. When the operating part 420 rotates relative to the docking base 221, the operating part 420 may push the sliding part 410 to move along the second direction Y through the sliding cooperation between the slide shaft 421 and the slide groove 461. By way of example, the slide shaft 421 may be provided on the operating rod 423, and the slide groove 461 may be provided the sliding part 410. Specifically, the operating part 420 may include the operating rod 423, and the slide shaft 421 may be provided at an end of the operating part 420 away from the actuating part 422.

In some embodiments, the sliding part 410 may include a push rod 460. When the operating part 420 rotates relative to the docking base 221, the operating part 420 may push the push rod 460 to switch the sliding part 410 from the blocking position to the releasing position. Specifically, the slide shaft 421 may be provided on the operating part 420, and the slide shaft 421 may be provided on the push rod 460 on the sliding part 410. Alternatively, the slide shaft 421 may be provided on the operating part 420, and the slide shaft 421 may be provided on the push rod 460 on the sliding part 410. By way of example, the slide groove 461 may be provided on the push rod 460.

In some embodiments, the operating part 420 may be provided on a side of the docking base 221 along the second direction Y for convenient operation.

In some embodiments, a fixing seat 450 may be provided on the docking base 221. The operating part 420 may be rotationally connected to the fixing seat 450 around an axis in the first direction X, such that the operating part 420 may be connected to the docking base 221 through the fixing seat 450.

In some embodiments, the push rod 460 may be connected to the linkage part 440 on a side of the linkage part 440 along the second direction Y. An end of the push rod 460 away from the linkage part 440 may extend through the fixing seat 450 and abuts against the operating part 420. When the operating part 420 rotates, the operating part 420 may push the end of the push rod 460 away from the linkage part 440, thereby pushing the push rod 460 to move along the second direction Y. Specifically, one of the operating part 420 and the end of the push rod 460 away from the linkage part 440 may be provided with the slide shaft 421, and the other of the operating part 420 and the end of the push rod 460 away from the linkage part 440 may be provided with the slide groove 461.

In some embodiments, the docking base 221 may be provided with a connecting seat 414, which may be located on the side of the linkage part 440 along the second direction Y. One end of the second elastic member 411 may be connected to the connecting seat 414, and the other end of the second elastic member 411 may be connected to the linkage part 440.

In some embodiments, the operating part 420 may be located on the side of the linkage part 440 along the second direction Y, and the second elastic member 411 may be positioned on a side of the linkage part 440 facing away from the operating part 420.

When the bed-side connector 331 is docked with the imaging-side connector 222, the ejector part 430 is in the return position. When it is necessary to detach the bed-side connector 331 from the imaging-side connector 222, the user may rotate the operating part 420 to switch the sliding part 410 to the release position. After the sliding part 410 switches to the release position, the sliding part 410 continues to move along the second direction Y away from the blocking position, thereby driving the linkage part 440 to move synchronously. During this movement, the linkage surface 441 synchronously moves away from the blocking position along the second direction Y. Due to the inclination of the linkage surface 441 from the end near the medical bed 300 to the end away from the medical bed 300, towards the direction from the blocking position to the release position, the synchronous movement of the linkage surface 441 along the second direction Y away from the blocking position may push the ejector part 430 to move along the first direction X, thereby switching the ejector part 430 from the return position to the eject position. During this process of switching from the return position to the eject position, the ejector part 430 may push the medical bed 300 (e.g., the supporting main body 320) to detach the bed-side connector 331 from the imaging-side connector 222. Thus, the operation of detaching the bed-side connector 331 from the imaging-side connector 222 is very convenient.

When the bed-side connector 331 is detached from the imaging-side connector 222, after releasing the operating part 420, an elastic restoring force of the first elastic member 411 drives the sliding part 410 to switch from the release position to the blocking position. Simultaneously, an elastic restoring force of the second elastic member 431 drives the ejector part 430 to switch from the eject position to the return position. During the process in which the elastic restoring force of the first elastic member 411 drives the sliding part 410 from the release position to the blocking position, the linkage part 440 moves synchronously with the sliding part 410. When the bed-side connector 331 of the medical bed 300 docks again with the imaging-side connector 222 of the imaging device 200, the limiting part 223 may block the tensioning part 334 from moving away from the imaging main body 210 along the first direction X, and the ejector part 430 may yield to the medical bed 300 (e.g., the supporting main body 320). The medical bed 300 (e.g., the supporting main body 320) moves the bed-side connector 331 to a position where the bed-side connector 331 docks with the imaging-side connector 222.

Due to uneven ground and other reasons, it is often difficult to accurately align the bed-side connector 331 of the docking structure 330 of the medical bed 300 with the imaging-side connector 222 of the connection structure 220 of the imaging device 200, resulting in significant difficulty in docking. Some embodiments of the present disclosure provide a floating structure 500. At least one of the bed-side connector 331 or the imaging-side connector 222 adopts the floating structure 500, thereby reducing the difficulty of docking between the bed-side connector 331 and the imaging-side connector 222.

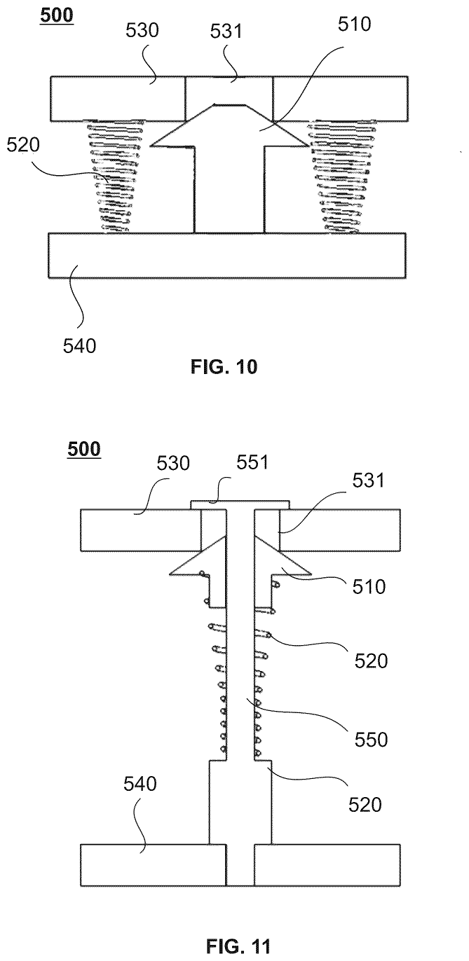

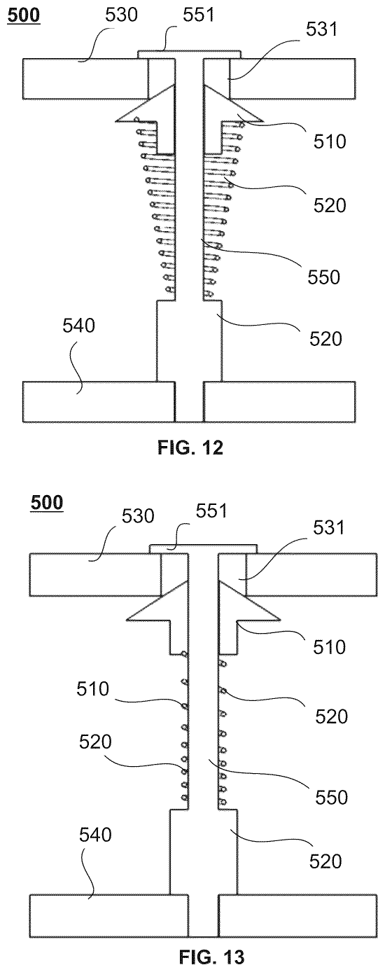

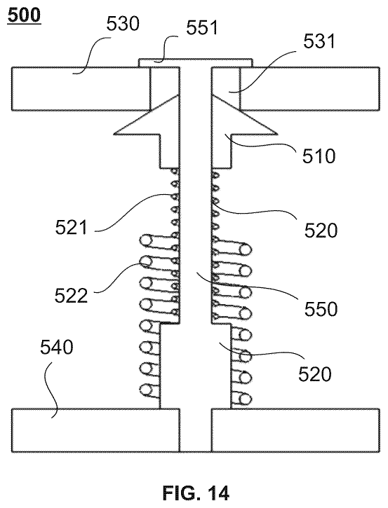

FIG. 10 is a schematic diagram of an exemplary structure of a floating structure according to some embodiments of the present disclosure. FIG. 11 is a schematic diagram of another exemplary structure of the floating structure according to some embodiments of the present disclosure. FIG. 12 is a schematic diagram of another exemplary structure of the floating structure according to some embodiments of the present disclosure. FIG. 13 is a schematic diagram of another exemplary structure of the floating structure according to some embodiments of the present disclosure. FIG. 14 is a schematic diagram of another exemplary structure of the floating structure according to some embodiments of the present disclosure. The following will describe a floating structure 500 in conjunction with FIGS. 10 to 14.

As shown in FIG. 10, the floating structure 500 includes a cone portion 510, an elastic structure 520, a first plate 530, and a second plate 540. The first plate 530 may be provided with a through hole 531, and the cone portion 510 may abut against the through hole 531. The elastic structure 520 may be located between the first plate 530 and the second plate 540, and an elastic force of the elastic structure 520 tends to move the cone portion 510 towards a direction of insertion into the through hole 531, thereby causing an outer peripheral surface of the cone portion 510 to taper along the direction of insertion into the through hole 531. An elastic coefficient of the elastic structure 520 may be a variable value. For example, in a direction from the second plate 540 towards the first plate 530, the elastic coefficient of the elastic structure 520 may increase, decrease, or fluctuate.

Since the elastic structure 520 is positioned between the first plate 530 and the second plate 540, the elastic force of the elastic structure 520 may cause the cone portion 510 to abut against a hole wall of the through hole 531. Therefore, when an external force acts on the floating structure 500, the first plate 530 and the second plate 540 moves closer together to compress the elastic structure 520, the cone portion 510 indirectly resists the elastic force of the elastic structure 520 through the external force, thereby allowing the cone portion 510 to move relative to the hole wall of the through hole 531. Additionally, due to the tapered structure of the outer peripheral surface of the cone portion 510 towards the direction of insertion into the through hole 531, the movement between the cone portion 510 and the hole wall of the through hole 531 is omnidirectional (i.e., movable in any direction), thereby permitting universal movement between the first plate 530 and the second plate 540. When the external force is removed, under an action of the elastic force of the elastic structure 520, the cone portion 510 returns to a position of insertion into the through hole 531 and abuts against the hole wall of the through hole 531, thereby restoring the first plate 530 and the second plate 540 to their specific relative positions.

In some embodiments, when the floating structure 500 is configured for the bed-side connector 331 or the imaging-side connector 222, one of the first plate 530 and the second plate 540 acts as a fixed plate while the other acts as a floating plate. The floating plate may move universally relative to the fixed plate, and an insertion module may be placed on a side of the floating plate facing away from the fixed plate. At least one of the bed-side connector 331 or the imaging-side connector 222 adopts the floating structure 500, facilitating the docking between the bed-side connector 331 and the imaging-side connector 222 through the universal movement of the floating plate relative to the fixed plate. Because the elastic coefficient of the elastic structure 520 varies in magnitude, a portion of the elastic structure 520 with a smaller elastic coefficient may be more prone to deformation. The cone portion 510 and the hole wall of the through hole 531 moves universally, thereby facilitating accurate alignment of the bed-side connector 331 with the imaging-side connector 222 during docking. A portion of the elastic structure 520 with a greater elastic coefficient provides a greater elastic force, thereby ensuring sufficient force between the bed-side connector 331 and the imaging-side connector 222 to reliably complete the docking. In summary, the design of the floating structure 500 reduces the difficulty of docking between the bed-side connector 331 and the imaging-side connector 222, thereby enhancing the reliability of docking.

In some embodiments, in a direction from the second plate 540 towards the first plate 530, the elastic coefficient of the elastic structure 520 decreases. In other words, the closer the elastic structure 520 is to the cone portion 510 and the through hole 531, the smaller the elastic coefficient of the elastic structure 520 is, making it easier for the elastic structure 520 to deform. The cone portion 510 and the hole wall of the through hole 531 moves universally, thereby facilitating accurate alignment of the bed-side connector 331 with the imaging-side connector 222 during docking. Conversely, in a direction away from the first plate 530 towards the second plate 540, the elastic coefficient of the elastic structure 520 varies. In other words, the closer the elastic structure 520 is to the second plate 540, the greater the elastic coefficient of the elastic structure 520 is, and a greater elastic force between the first plate 530 and the second plate 540 is provided. Thus ensuring sufficient force between the bed-side connector 331 and the imaging-side connector 222 to reliably complete the insertion.

The elastic structure 520 may be a helical spring structure or an elastic column structure. In the direction from the second plate 540 towards the first plate 530, the elastic coefficient of the elastic structure 520 varies, which may be gradually varying or segmentally varied.

In some embodiments, the elastic structure 520 may include an integrally formed helical spring, and the elastic coefficient of the helical spring varies, which may be gradually varying or segmented. Preferably, in the direction from the second plate 540 towards the first plate 530, the elastic coefficient of the helical spring decreases.

In some embodiments, one or more of a diameter of a spring coil, a wire diameter, an elastic modulus of a material, an effective count of levels, or any combination thereof of the helical spring are variable values, so that the elastic coefficient of the helical spring is a variable value.

In some embodiments, a pitch of the helical spring may be a variable value, so that the elastic coefficient of the helical spring is a variable value. For example, in the direction from the second plate 540 towards the first plate 530, the pitch of the helical spring increases, as shown in FIG. 13.

In some embodiments, an outer diameter of the helical spring may be a variable value, so that the elastic coefficient of the helical spring is a variable value. For example, in the direction from the second plate 540 towards the first plate 530, the outer diameter of the helical spring increases, as shown in FIG. 12.

In some embodiments, both the outer diameter and the pitch of the helical spring are variable values, so that the elastic coefficient of the helical spring is a variable value. For example, in the direction from the second plate 540 towards the first plate 530, both the outer diameter and the pitch of the helical spring increase, as shown in FIG. 11.

In some embodiments, the elastic structure 520 may include a plurality of springs arranged sequentially. At least one of the plurality of springs spring has a different elastic coefficient compared to the rest of the plurality of springs, so that the elastic coefficient of the helical spring is a variable value, as shown in FIG. 14.

Referring to FIG. 14, in some embodiments, the elastic structure 520 may include a plurality of springs arranged sequentially, where the elastic coefficient of the plurality of springs varies sequentially in the direction from the second plate 540 towards the first plate 530. In some embodiments, in the direction from the second plate 540 towards the first plate 530, the elastic coefficient of the plurality of springs decreases sequentially.

In some embodiments, the plurality of springs may be non-integrally formed, and connected after separate formation. By sequentially varying the elastic coefficient of the sequentially connected plurality of springs, it is also possible to achieve a variable elastic coefficient for the elastic structure 520 in the direction from the second plate 540 towards the first plate 530.

In some embodiments, a count of levels of the plurality of springs varies sequentially in the direction from the second plate 540 towards the first plate 530. Because the count of levels of the plurality of springs varies sequentially, the elastic coefficient of the plurality of springs is a variable value. In some embodiments, in the direction from the second plate 540 towards the first plate 530, the count of levels of the plurality of springs decreases sequentially. By way of example, the elastic structure 520 may include a first-level spring 521 and a second-level spring 522. The first-level spring 521 is located on a side of the second-level spring 522 closer to the first plate 530, thereby achieving a sequential decrease in the elastic coefficient of the plurality of springs in the direction from the second plate 540 towards the first plate 530.

In some embodiments, the elastic coefficient of the plurality of springs varies sequentially in the direction from the second plate 540 towards the first plate 530, which may be achieved by varying the pitch of the plurality of springs or by varying the outer diameter of the plurality of springs, or by varying both the pitch and outer diameter of the plurality of springs.

Referring to FIGS. 11 to 14, in some embodiments, the floating structure 500 may further include a guide rod 550. One end of the guide rod 550 may be fixedly connected to the second plate 540, and the other end of the guide rod 550 may be connected to a limiting protrusion 551 after passing through the through-hole 531. A size of the limiting protrusion 551 may be greater than a size of the through-hole 531, so the limiting protrusion 551 may abut against the first plate 530 to prevent the first plate 530 from detaching from the guide rod 550. When the external force is removed, under the elastic force of the elastic structure 520, the first plate 530 moves towards the direction away from the second plate 540 until the first plate 530 abuts against the limiting protrusion 551, thereby maintaining the first plate 530 and the second plate 540 in their respective relative positions.

In some embodiments, the cone portion 510 may be connected to the guide rod 550, and the elastic structure 520 may be sleeved on the guide rod 550. Sleeving the elastic structure 520 on the guide rod 550 facilitates guidance for the elastic force of the elastic structure 520. Thus, when the external force is removed, under the elastic force of the elastic structure 520, the cone portion 510 may quickly return to a position for insertion into the through-hole 531 and abut against the wall of the through-hole 531. The first plate 530 and the second plate 540 may quickly return to their respective relative positions.

In some embodiments, the cone portion 510 may be located on one side of the first plate 530 close to the second plate 540, so the direction of insertion of the cone portion 510 into the through-hole 531 is towards the first plate 530 as viewed from the second plate 540. The cone portion 510 may be movably sleeved on the guide rod 550, and the elastic structure 520 may be located between the cone portion 510 and the second plate 540.

In some embodiments, a side wall of the guide rod 550 near one end adjacent to the second plate 540 may be protruded to form an abutting portion 552. At least part of the elastic structure 520 may be located between the abutting portion 552 and the cone portion 510. A side of the cone portion 510 facing the second plate 540 may abut against the elastic structure 520, and a side of the abutting portion 552 facing the first plate 530 may abut against the elastic structure 520, thereby allowing the cone portion 510 to move along the guide rod 550 through the elastic deformation of the elastic structure 520.

In some embodiments, the entire elastic structure 520 may be located between the cone portion 510 and the abutting portion 552. One end of the elastic structure 520 may abut against the cone portion 510, and the other end of the elastic structure 520 may abut against the abutting portion 552.

In some embodiments, a first-level spring 521 of the elastic structure 520 may be located between the cone portion 510 and the abutting portion 552. One end of the first-level spring 521 may abut against the cone portion 510, and the other end of the first-level spring 521 may abut against the abutting portion 552. One end of a second-level spring 522 may be sleeved outside the first-level spring 521, and the other end of the second-level spring 522 away from the first-level spring 521 may be connected to the second plate 540.

In some embodiments, the abutting portion 552 may not be provided, and the elastic structure 520 may directly abut against the second plate 540 at the end away from the cone portion 510, enabling the cone portion 510 to move along the guide rod 550 through the elastic deformation of the elastic structure 520.

In some embodiments, the cone portion 510 may be located on the side of the first plate 530 away from the second plate 540, so the direction of insertion of the cone portion 510 into the through-hole 531 is towards the second plate 540 as viewed from the first plate 530. The cone portion 510 may be fixedly connected to an end of the guide rod 550 away from the second plate 540. Two ends of the elastic structure 520 may abut against the first plate 530 and the second plate 540, respectively. Thus, when the first plate 530 moves closer to the second plate 540, the cone portion 510 moves outward relative to the through-hole 531. Due to the tapered outer peripheral surface of the cone portion 510 along the direction of insertion into the through-hole 531, there is a gap between the tapered outer surface of the cone portion 510 and the wall of the through-hole 531, allowing universal movement between the cone portion 510 and the wall of the through-hole 531, thus enabling universal movement between the first plate 530 and the second plate 540.

In some embodiments, along the direction of insertion of the cone portion 510 into the through-hole 531, the hole wall of the through-hole 531 tapers, so that a trend of variation of the hole wall of the through-hole 531 matches a trend of variation of the outer peripheral surface of the cone portion 510. Thus, the tapered outer peripheral surface of the cone portion 510 may fit well with the hole wall of the through-hole 531, and when external force is removed, under the elastic force of the elastic structure 520, the cone portion 510 may return to the position of insertion into the through-hole 531, and the tapered outer peripheral surface of the cone portion 510 may fit well with the hole wall of the through-hole 531.

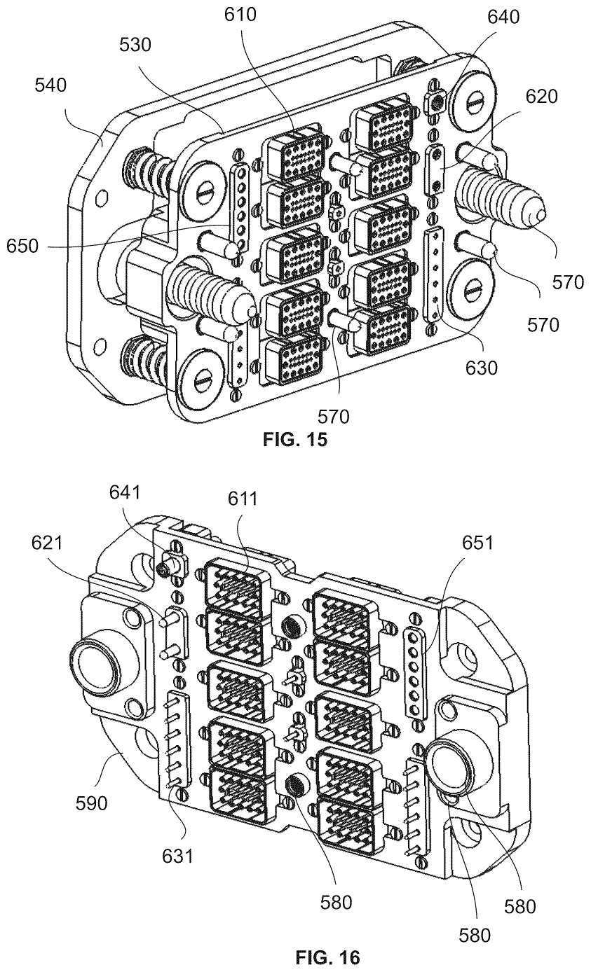

FIG. 15 is a schematic diagram of an exemplary structure of a floating connector according to some embodiments of the present disclosure, and FIG. 16 is a schematic diagram of an exemplary structure of another floating connector that mates with the floating connector shown in FIG. 15.

Referring to FIG. 15, some embodiments of the present disclosure provide a floating connector. The floating connector may include the floating structure 500 described in any of the above embodiments, and both the first plate 530 and the second plate 540. One of the first plate 530 or the second plate 540 may have a plug-in module on a side facing away from the other one of the first plate 530 or the second plate 540. One of the first plate 530 or the second plate 540 that is provided with the plug-in module may be referred to as a floating plate, while the other one of the first plate 530 or the second plate 540 may be referred to as a fixed plate. The plug-in module may include any one of a radio frequency module 610, a power module 620, an electrical signal module 630, a liquid module 640, an optical fiber module 650, or any combination thereof. The first plate 530 may be the fixed plate, and the second plate 540 may be the floating plate. Alternatively, the second plate 540 may be the fixed plate, and the first plate 530 may be the floating plate.

At least one of the medical bed 300 of the medical imaging system 140 and the imaging device 200 adopts the floating connector of this embodiment. Through a universal movement of the floating plate relative to the fixed plate, the alignment of the plug-in module of the floating connector of the medical bed 300 with the plug-in module of the floating connector of the imaging device 200 is facilitated. Due to varying elastic coefficients of the elastic structure 520, a portion of the elastic structure 520 with a relatively small elastic coefficient may be easier to deform, which allows the cone part 510 and the hole wall of the through-hole 531 to move universally, thereby facilitating the alignment of the plug-in module of the floating connector of the medical bed 300 with the plug-in module of the floating connector of the imaging device 200 during mating. A portion of the elastic structure 520 with a relatively large elastic coefficient may provide a greater elastic force, which ensures that sufficient insertion force is provided between the plug-in module of the floating connector of the medical bed 300 and the plug-in module of the floating connector of the imaging device 200, thereby enabling reliable insertion. In summary, this configuration not only facilitates the alignment of the plug-in module of the floating connector of the medical bed 300 with the plug-in module of the floating connector of the imaging device 200 during mating, but also ensures reliable insertion.

The floating connector described above can be a male connector for insertion into a matching female connector. The floating connector may also be a female connector configured to receive a matching male connector.

The medical bed 300 or the imaging device 200 may adopt the above described floating connector. Alternatively, both the medical bed 300 and the imaging device 200 may adopt the above described floating connector.

The floating connector with the floating structure 500 as shown in FIG. 15 in any of the aforementioned embodiments may be referred to as a first connector. As shown in FIG. 16, the embodiments of the present disclosure also provide another floating connector, referred to as a second connector. The second connector may be configured to mate with the first connector. The second connector may include a connecting plate 590. The connecting plate 590 may be provided with a plug-in module, which may include any one of a radio frequency module 611, a power module 621, an electrical signal module 631, a liquid module 641, an optical fiber module 651, or any combination thereof. The radio frequency module 610 of the plug-in module of the first connector may be configured to mate with the radio frequency module 611 of the plug-in module of the second connector to achieve a radio frequency conduction. The power module 620 of the plug-in module of the first connector may be configured to mate with the power module 621 of the plug-in module of the second connector to achieve a power conduction. The electrical signal module 630 of the plug-in module of the first connector may be configured to mate with the electrical signal module 631 of the plug-in module of the second connector to achieve an electrical signal conduction. The liquid module 640 of the plug-in module of the first connector may be configured to mate with the liquid module 641 of the plug-in module of the second connector to achieve a liquid conduction. The optical fiber module 650 of the plug-in module of the first connector may be configured to mate with the optical fiber module 651 of the plug-in module of the second connector to achieve an optical conduction. The first connector and the second connector may be used for the medical bed 300 and the imaging device 200 respectively, thereby enabling the docking of the medical bed 300 with the imaging device 200.

Referring to FIG. 15, in some embodiments, the floating structure of the first connector may further include a guide element 570, with one end of the guide element 570 connected to the fixed plate and another end of the guide element 570 passing through the floating plate. The connecting plate 590 of the second connector may be provided with a guide coupling structure 580 that mates with the guide element 570. The guide element 570 and the guide coupling structure 580 may be movably fitted, and a direction of their movement corresponds to a mating direction of the plug-in module of the first connector and the plug-in module of the second connector. Therefore, a movable fit between the guide element 570 and the guide coupling structure 580 facilitates the accurate alignment of the plug-in module of the first connector and the plug-in module of the second connector.

In some embodiments, the guide element 570 may be a guide pin, and the guide coupling structure 580 may be a guide sleeve.

In some embodiments, the guide element 570 may be a guide sleeve, and the guide coupling structure 580 may be a guide pin.

In some embodiments, the floating structure 500 may include multi-level guide elements 570. The higher a level of a guide element 570 is, the shorter a length of the guide element 570 and the smaller a diameter of the guide element 570 may be. The length of the guide element 570 corresponds to the relative movement direction between the guide coupling structure 580 and the guide element 570. Correspondingly, the second connector may include multi-level guide coupling structures 580. The guide coupling structures 580 may correspond to the guide elements 570 in a one-to-one correspondence. The higher a level of a guide coupling structure 580 is, the shorter a length of the guide coupling structure 580 and the smaller a diameter of the guide coupling structure 580 may be. By providing the multi-level guide elements 570, multi-level guidance between the first connector and the second connector can be achieved.

In some embodiments, any sub-module of the plug-in module may be detachably connected to the floating plate, thereby making it easy to replace.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

As another example, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This way of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numerical parameter set forth in the written description and attached claims are approximations that may vary depending upon the desired properties sought to be obtained by a particular embodiment. In some embodiments, the numerical parameter should be construed in light of the number of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A docking structure installed on a medical bed, the docking structure being configured to dock with a connection structure of an imaging device to achieve docking between the medical bed and the imaging device, wherein the docking structure comprises an electric driving assembly, the electric driving assembly being provided on the medical bed, and the electric driving assembly being at least configured to drive the medical bed to move towards the imaging device, so as to achieving docking between the docking structure and the connection structure, and achieving docking between the medical bed and the imaging device.

2. The docking structure of claim 1, wherein the docking structure comprises a mounting seat, wherein a tensioning part is provided on the mounting seat, the tensioning part has a locked position and an unlocked position, the tensioning part is configured to fit with a limiting part of the connection structure to lock or unlock a movement of the mounting seat, and an elastic part is provided between the tensioning part and the mounting seat, the elastic part being configured to restore the tensioning part from the unlocked position to the locked position;
the docking structure further comprises a bed-side connector, the bed-side connector being provided at one end of the medical bed that docks with the imaging device, and the electric driving assembly being drivingly connected to the mounting seat.

3. The docking structure of claim 2, wherein the electric driving assembly is at least configured to: when the tensioning part is in the locked position and cooperates with the limiting part of the connection structure, the electric driving assembly drives the medical bed to move towards the mounting seat, so as to cause the medical bed to move relative to the mounting seat, thereby achieving docking between the bed-side connector and the connection structure, and achieving docking between the medical bed and the imaging device.

4. The docking structure of claim 3, wherein the docking structure comprises a tensioning assembly, the tensioning assembly including a rotating shaft provided on the mounting seat, a pull rod being rotatably connected to the rotating shaft, and one end of the pull rod being provided with the tensioning part.

5. The docking structure of claim 4, wherein the docking structure further comprises a supporting part, the supporting part is connected to the medical bed, the bed-side connector is provided on the supporting part, a stopping part is provided on the supporting part, a projection of the stopping part at least partially overlaps with an end of the pull rod away from the tensioning part in a first direction, and the first direction being a length direction of the medical bed.

6. The docking structure of claim 5, wherein the tensioning assembly includes two pull rods arranged at interval in a second direction, the second direction is a width direction of the medical bed, the two pull rods are rotatably connected to the rotating shaft, the two pull rods are provided with the tensioning part, respectively, each end of the two pull rods away from the corresponding tensioning part is connected by a connecting rod, and the projection of the stopping part at least partially overlaps with the connecting rod in the first direction.

7. The docking structure of claim 1, wherein the electric driving assembly includes a driving source, in a docking state, the driving source is arranged at a position where a magnetic field strength of a magnet of the imaging device is 5 mT-200 mT.

8. The docking structure of claim 7, wherein the electric driving assembly further includes a moving structure, the driving source is arranged on the moving structure, and the moving structure is configured to adjust a position of the driving source, thereby the driving source is arranged at positions corresponding to different magnetic field strengths of the magnet of the imaging device in the docking state.

9. The docking structure of claim 7, wherein in the docking state, a distance between the driving source and the magnet facing one side of the medical bed is positively correlated with a maximum magnetic field strength of the magnet.

10. The docking structure of claim 1, wherein the docking structure is provided with a sensor, the sensor being configured to measure an orientation of the docking structure relative to the connection structure of the imaging device, the orientation including a direction and/or a distance of the docking structure relative to the connection structure of the imaging device.

11. The docking structure of claim 10, wherein the sensor is connected to a display device via a signal, the display device being configured to display a measurement result of the sensor.

12. The docking structure of claim 2, wherein the docking structure further comprises a guiding part connected to the bed-side connector, the guiding part is configured to fit with a guidance part of the connection structure to guide the docking of the bed-side connector with an imaging-side connector of the connection structure.

13. (canceled)

14. The docking structure of claim 2, wherein the bed-side connector further includes a height adjustment mechanism, the height adjustment mechanism being configured to adjust a height of the bed-side connector relative to the connection structure of the imaging device.

15. A docking method for an imaging device and a medical bed including a docking structure, the docking structure being installed on the medical bed, the docking structure being configured to dock with a connection structure of the imaging device to achieve docking between the medical bed and the imaging device, wherein the docking structure comprises an electric driving assembly, the electric driving assembly being provided on the medical bed, and the electric driving assembly being at least configured to drive the medical bed to move towards the imaging device, so as to achieving docking between the docking structure and the connection structure, and achieving docking between the medical bed and the imaging device;
the docking method comprising:
driving the medical bed to move towards the imaging device to a first critical position, wherein when the medical bed is at the first critical position, a limiting part of the connection structure just contacts a tensioning part of the docking structure, and the tensioning part is in a locked position;
driving the medical bed to continue moving to a second critical position, wherein when the tensioning part gradually moves from the locked position to an unlocked position, an elastic part of the docking structure gradually stretches, and when the medical bed is at the second critical position, the tensioning part is in the unlocked position;
driving the medical bed to continue moving to a third critical position, wherein the tensioning part moves across the limiting part and is restored from the unlocked position to the locked position under an action of the elastic part, and when the medical bed is at the third critical position, the tensioning part hooks with the limiting part; and
initiating the electric driving assembly to drive a mounting seat of the docking structure to move relative to the medical bed, so that the medical bed docks with the imaging device.

16. The docking method of claim 15, wherein the docking structure and/or the medical bed is provided with a sensor, the medical bed is provided with a processor, and the driving the medical bed to move towards the imaging device to the first critical position includes:
automatically adjusting a movement parameter of the medical bed by the processor based on a measurement result of the sensor.

17. The docking method of claim 16, wherein the movement parameter of the medical bed includes at least one of a moving direction, a moving speed, or a height of the medical bed relative to the ground.

18. The docking method of claim 16, wherein the bed-side connector further includes a height adjustment mechanism, and the driving the medical bed to move towards the imaging device to the first critical position includes:
controlling, by the processor, based on the measurement result of the sensor, the height adjustment mechanism to adjust a height of the bed-side connector.

19. The docking method of claim 15, wherein when the medical bed is at the third critical position, the tensioning part hooks with the limiting part to lock the movement of the mounting seat, and the initiating the electric driving assembly to drive the mounting seat to move relative to the medical bed, so that the medical bed docks with the imaging device includes:
driving the mounting seat to move in a direction away from the imaging device by the electric driving assembly, so that a driving force acting on the mounting seat reacts against the medical bed, causing the medical bed to move towards the imaging device to complete the docking.

20. The docking method of claim 15, wherein the tensioning part is provided with a signal generator, the signal generator being configured to output a sensing signal when the tensioning part hooks with the limiting part, and the initiating the electric driving assembly to drive the mounting seat to move relative to the medical bed, so that the medical bed docks with the imaging device includes:
in response to receiving the sensing signal, initiating the electric driving assembly.

21. (canceled)

22. A docking detachment structure that is fit with a docking structure, the docking structure being installed on a medical bed, the docking structure being configured to dock with a connection structure of an imaging device to achieve docking between the medical bed and the imaging device, wherein the docking structure comprises an electric driving assembly, the electric driving assembly being provided on the medical bed, and the electric driving assembly being at least configured to drive the medical bed to move towards the imaging device, so as to achieving docking between the docking structure and the connection structure, and achieving docking between the medical bed and the imaging device;
wherein the docking detachment structure is provided on the connection structure, the docking detachment structure is configured such that: under an action of external force, the docking detachment structure causes the docking structure and the connection structure to separate along a first direction, and a direction of the external force is perpendicular to the first direction.

23-24. (canceled)
